SEQUENCE LISTING (7 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - RNA, 10 nt, Artificial Sequence
  Description of Artificial Sequence Synthetic oligonucleotide
gccrccaugg

SEQ ID NO: 4 - RNA, 13 nt, Artificial Sequence
  Description of Artificial Sequence Synthetic oligonucleotide
gccgccrccaugg

SEQ ID NO: 5 - RNA, 300 nt, Artificial Sequence
  Description of Artificial Sequence Synthetic polynucleotide
  misc_feature (1)..(300): This sequence may encompass 20-300 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 100 nt, Artificial Sequence
  Description of Artificial Sequence Synthetic polynucleotide
  misc_feature (1)..(100): This sequence may encompass 95-100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - protein, 7 aa, Severe acute respiratory syndrome coronavirus 2
QTQTNSP

(19) United States
(12) Patent Application Publication
(10) Pub. No.: US 2023/0043887 A1
(43) Pub. Date: Feb. 9, 2023

(54) COMPOSITIONS AND METHODS RELATED TO SURGE-ASSOCIATED SARS-COV-2 MUTANTS

(71) Applicants: Venkataramanan Soundararajan, Andover, MA (US); Aiveliagaram Venkatakrishnan, Cambridge, MA (US); Joseph Du-Che Yao, Rochester, MN (US)

(72) Inventors: Venkataramanan Soundararajan, Andover, MA (US); Aiveliagaram Venkatakrishnan, Cambridge, MA (US); Joseph Du-Che Yao, Rochester, MN (US)

(21) Appl. No.: 17/750,817

(22) Filed: May 23, 2022

Related U.S. Application Data
(60) Provisional application No. 63/192,434, filed on May 24, 2021.

(51) Int. Cl. A61K 39/215; C07K 16/10; C12Q 1/70

(52) U.S. Cl. CPC A61K 39/215; C07K 16/10; C12Q 1/70

(57) ABSTRACT

Compositions for use as a vaccine against SARS-CoV-2 infection are disclosed, which comprise either a polypeptide that comprises at least one surge-associated mutation (e.g., deletion) in its amino acid sequence or a nucleic acid (e.g., mRNA) that encodes said polypeptide. Also disclosed are formulations that include these compositions, antibodies or their antigen-biding fragments directed to these polypeptides, methods of making such antibodies, methods of vaccinating subjects against SARS-CoV-2 infection, and methods of selecting an antibody, convalescent plasma, or vaccine against SARS-CoV-2 infection.

### CROSS-REFERENCE TO RELATED APPLICATIONS

This application claims the benefit of U.S. Provisional Application No. 63/192,434, filed May 24, 2021, which is hereby incorporated by reference in its entirety.

### SEQUENCE LISTING

The instant application contains a Sequence Listing which has been filed electronically in ASCII format and is hereby incorporated by reference in its entirety. Said ASCII copy, created on Aug. 8, 2022, is named LBH-02001_SL.txt and is 18,144 bytes in size.

### BACKGROUND

The ongoing COVID-19 pandemic has infected around 500 million people and killed more than 6.1 million people worldwide, as of April 2022. The continual emergence of SARS-CoV-2 variants with increased transmissibility and capacity for immune escape, such as B.1.17 (“UK variant”) and P.1 (“Brazilian variant”), threatens to prolong the pandemic through devastating outbreaks such as the one currently being witnessed in India.

While multiple vaccines have demonstrated high effectiveness in clinical trials and real-world studies, there have been reports of “vaccine breakthrough infections” with SARS-CoV-2 variants. A recent study described two such cases in New York, at least one of which occurred despite confirmation of a robust neutralizing antibody response. Variant classification schemes have been developed by the US Centers for Disease Control and Prevention (CDC) and the World Health Organisation (WHO) based on factors such as prevalence, evidence of transmissibility and disease severity, and ability to be neutralized by existing therapeutics or sera from vaccinated patients.

It is imperative to further understand and combat these emerging Variants of Concern/Interest to contain the ongoing pandemic and manage or prevent future outbreaks.

### SUMMARY OF THE INVENTION

In some aspects, compositions for use as a vaccine against SARS-CoV-2 infection comprise either a polypeptide that comprises at least one surge-associated mutation in its amino acid sequence with respect to SEQ ID NO: 1 or a nucleic acid that encodes said polypeptide.

In some embodiments, said at least one mutation is within the residue range 13-303 of SEQ ID NO: 1. In preferred embodiments, said at least one mutation is a deletion.

In some embodiments, the composition comprises said nucleic acid (e.g., ribonucleic acid). In some embodiments, the composition comprises a messenger ribonucleic acid (mRNA). In some embodiments, the mRNA comprises a 5′ cap, 5′-untranslated region, a 3′-untranslated region, and a poly(A) tail. In some embodiments, the mRNA acid comprises at least one non-canonical nucleobase.

In some embodiments, said mutation is a deletion of any one or more residues selected from 14-16, 24-26, 63-76, 81, 85-89, 136-146, 150-165, 167-169, 210-212, 214, 216 and 241-252.

In some embodiments, said mutation is a deletion of two or more residues selected from 14-16, 24-26, 63-76, 81, 85-89, 136-146, 150-165, 167-169, 210-212, 214, 216 and 241-252. In some embodiments, said mutation is a deletion of 3, 4, 5, 6, 7, 8, 9, or 10 residues selected from 14-16, 24-26, 63-76, 81, 85-89, 136-146, 150-165, 167-169, 210-212, 214, 216 and 241-252. In some embodiments, the mutation comprises a contiguous stretch of residues. In some embodiments, the mutation comprises two separate contiguous stretches of residues. In some embodiments, the mutation comprises three or more separate contiguous stretches of residues. In certain preferred embodiments, said mutation is a deletion of one or more residues selected from those described in FIGS. 1 to 6 and Tables 1 to 5 and the Example. In some embodiments, said polypeptide also has K986P and V987P mutations. In some embodiments, said polypeptide has at least one additional mutation selected from E484K, N501Y, D614G, P681H, and P681R.

In some aspects, compositions comprise two or more of the polypeptides described above, or nucleic acids that encode two or more of the polypeptides.

In some aspects, antibodies or antigen-binding fragments thereof are disclosed, which bind to the polypeptides described above. Suitable methods can be used to generate such antibodies against the disclosed polypeptides.

In some aspects, formulations comprise the compositions described above or elsewhere herein. In some such embodiments, the formulations comprise at least one excipient. For example, the formulations further comprise a delivery system. In some such embodiments, the delivery system is selected from protamine, protamine liposome, polysaccharide particles, cationic nanoemulsion, cationic polymer, cationic polymer liposome, cationic lipid nanoparticle, cationic lipid/cholesterol nanoparticles, cationic lipid/cholesterol/PEG nanoparticles, and dendrimer nanoparticles.

In some aspects, formulations comprise two or more of the polypeptides (or nucleic acids encoding two or more of the polypeptides) described above or elsewhere herein and at least one excipient.

In some aspects, methods of vaccinating a subject against SARS-CoV-2 infection comprise administering to the subject a composition or a formulation as described above or elsewhere herein. In some such embodiments, said administering is via intramuscular injection or intradermal injection.

In some aspects, methods of selecting an antibody for treating a SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a SARS-CoV-2 spike protein from the subject; and selecting an antibody that does not bind to the N-terminal domain antigenic supersite of the SARS-CoV-2 spike protein.

In some aspects, methods of making an antibody comprise using a polypeptide described above or elsewhere herein as the target antigen.

In some aspects, methods of selecting a convalescent plasma against SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a SARS-CoV-2 spike protein from the subject; and selecting a convalescent plasma having antibodies that do not bind to the N-terminal domain antigenic supersite of the SARS-CoV-2 spike protein.

In some aspects, methods of selecting a vaccine against SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a spike protein from an emerging variant of SARS-CoV-2; and selecting a vaccine having a polypeptide described above or elsewhere herein.

Further embodiments and details for each of these aspects is presented throughout the disclosure.

### BRIEF DESCRIPTION OF THE FIGURES

FIGS. 1A-C. Identifying potential SARS-CoV-2 variants contributing to COVID-19 surge. (FIG. 1A) Overview of COVID-19 prevalence and SARS-CoV-2 variants globally during the pandemic. (FIG. 1B) Correlations between mutational prevalence and test positivity over three-month windows for each country (e.g. India). (FIG. 1C) Correlations between mutational prevalence and test positivity and mutational prevalence over all three-month time windows that have a surge in test positivity. The enrichment of deletions among surge-associated mutations.

FIGS. 2A-B. Rapidly emerging deletions in India map to antigenic supersite in N-terminal domain mapping to antigenic supersite. (FIG. 2A) Identification of mutations in the SARS-CoV-2 Spike protein that are associated with the COVID-19 surge in India between February and April 2021, based on correlations between mutational prevalence and test positivity. 13 mutations were found to be positively correlated with the surge over the three-month window. (FIG. 2B) Tracking the prevalence of ΔF157/R158 in the emerging “Indian variant”. The inset shows the location of these two residues on the Spike protein structure.

FIGS. 3A-C. Emerging Chile variant has uncharacterized deletions mapping to antigenic supersite. (FIG. 3A) Identification of mutations in the SARS-CoV-2 Spike protein that are associated with the COVID-19 surge in Chile between February and April 2021, based on correlations between mutational prevalence and test positivity. 36 mutations were found to be positively correlated with the surge over the three-month window. The clustering of these mutations based on their co-occurrence (number of sequences with both mutations present) using the complete method (farthest point algorithm) reveals two dominant clusters. (FIG. 3B) Deletions in the emerging “Chile variant” map to the antigenic supersite that is bound by most anti-NTD antibodies. (FIG. 3C) Rapid increase of the prevalence of surge-associated mutations in the Chile variant (blue). The other existing variants (B.1.1.7, P.2) are also shown.

FIGS. 4A-C. Deletion mutations present in the Spike proteins sequences derived from vaccination breakthrough or reinfection cases. (FIG. 4A) NGS of SARS-CoV-2 RNA from COVID-19 vaccine breakthrough or reinfection cases from the Mayo Clinic. (FIG. 4B) Four different stretches of Spike protein deletions from the patients with vaccine breakthrough or reinfections of COVID-19. In the heatmap, rows denote patients (state, date of sample and vaccination status are shown) and columns denote deletion mutations. Filled boxes denote the presence of deletion mutations, which are shown on the 3D structure of the Spike protein in panel C. (FIG. 4C) Positions corresponding to deletion mutations are shown as spheres.

FIGS. 5A-C. Deletion mutations are expanding to contiguous regions. (FIG. 5A) Frequency of occurrence of deletion mutations in the N-terminal domain across 9.3 million Spike protein sequences. The recurrent deletion regions, both known as well as new, are illustrated schematically and mapped on the structure of the Spike protein. (FIG. 5B) Heatmap showing the expansion of “deletable” regions in the course of the pandemic, where the rows denote residue positions in the Spike protein and columns denote the time course of the pandemic (in months). The boxes denote the frequency of the deletion mutation across the world in that month. The color of the boxes corresponds to a frequency of 1 to 100,000 sequences shown on a log 10 scale. These deletion mutations are shown on the 3D structure of the Spike protein. (FIG. 5C) Positions corresponding to deletion mutations are shown as spheres.

FIG. 6 illustrates the comparison of surge-associated mutations identified in this study and mutations present in variants of interest or concern as categorized by the CDC.

### DETAILED DESCRIPTION OF THE INVENTION

The present disclosure is based, at least in part, to the discovery that deletions in the Spike protein NTD that map to an antigenic supersite have emerged over the course of the pandemic are strongly associated with case surges and are present in a subset of vaccine breakthrough variants.

In accord with this discovery and further findings, in some aspects, compositions for use as a vaccine against SARS-CoV-2 infection are disclosed, which comprise either a polypeptide that comprises at least one surge-associated mutation (e.g., deletion) in its amino acid sequence with respect to SEQ ID NO: 1 (e.g., at its NTD) or a nucleic acid (e.g., mRNA) that encodes said polypeptide. The mutation is preferably a deletion of any one residue or more than one residue or a range of contiguous residues selected from 14-16, 24-26, 63-76, 81, 85-89, 136-146, 150-165, 167-169, 210-212, 214, 216 and 241-252 with respect to SEQ ID NO: 1.

Additional aspects include various formulations that include these compositions, antibodies or their antigen-biding fragments directed to these polypeptides, methods of making such antibodies, methods of vaccinating subjects against SARS-CoV-2 infection, and methods of selecting an antibody, convalescent plasma, or vaccine against SARS-CoV-2 infection.

In certain preferred embodiments, the compositions described herein are injectable compositions with one or more excipients and no other pathogens or biological materials.

In certain preferred embodiments, two or more of the disclosed polypeptides (or nucleic acids encoding them) can be combined, or the disclosed mutations can be combined in a multi-antigen polypeptide, for use as a multi-prong vaccine.

### Definitions

As used in the description, the words “a” and “an” can mean one or more than one. As used in the claims in conjunction with the word “comprising,” the words “a” and “an” can mean one or more than one. As used in the description, “another” can mean at least a second or more.

A “formulation” refers to a mixture of one or more of the polypeptides or nucleic acids described herein, or pharmaceutically acceptable salts or hydrates thereof, with other chemical components, such as physiologically acceptable carriers and excipients. The purpose of a formulation is to facilitate administration to an organism.

The term “pharmaceutically acceptable salt” includes salts derived from inorganic or organic acids or bases, including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluroacetic, trichloroacetic, naphthalene-2 sulfonic and other acids; or salts with metals such as sodium, potassium, lithium, calcium, magnesium, and aluminum.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein, a therapeutic that “prevents” a disorder or condition refers to an agent (e.g., compound) that, in a statistical sample, reduces the occurrence of the disorder or condition in the treated sample relative to an untreated control sample, or delays the onset or reduces the severity of one or more symptoms of the disorder or condition relative to the untreated control sample.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in “Remington's Pharmaceutical Sciences” by E. W. Martin.

The terms “animal”, “subject”, and “patient” as used herein include all members of the animal kingdom including, but not limited to, birds, mammals, animals (e.g., cats, dogs, horses, and swine) and humans.

In some descriptions, reference is made to SEQ ID NO: 1, which is provided below.

>sp|P0DTC2|SPIKE_SARS2 Spike glycoprotein OS = Severe acute
respiratory syndrome coronavirus 2 OX = 2697049 GN = S PE = 1 SV = 1
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQS1IAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTORNFYEPOIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT

### Polypeptides, Nucleic Acids, and Mutations

In some aspects, compositions for use as a vaccine against SARS-CoV-2 infection comprise either a polypeptide that comprises at least one surge-associated mutation in its amino acid sequence with respect to SEQ ID NO: 1 or a nucleic acid that encodes said polypeptide.

SEQ ID NO: 1 is representative of the spike protein of SARS-CoV-2. Its N-terminal domain (NTD), according to the corresponding UniProt entry, is comprised of residues 13-303 of SEQ ID NO: 1. In some embodiments, the referenced mutations are in the NTD.

The mutations in some preferred embodiments are deletions. The deletions can be at any one residue, or at more than one residue (contiguous or not), which can be selected from the following set of residues: 14-16, 24-26, 63-76, 81, 85-89, 136-146, 150-165, 167-169, 210-212, 214, 216 and 241-252 with respect to SEQ ID NO: 1. In some embodiments, these selected mutations result in a spike protein with an altered NTD that does not bind to some antibodies that bind the NTD of SEQ ID NO: 1; therefore, use of such polypeptides allows creating a vaccine against emerging strains against which current therapies are not effective. In some embodiments, the polypeptide has additional mutations, such as K986P and V987P, and/or E484K, N501Y, D614G, P681H, and/or P681R.

In certain aspects, compositions include more than one polypeptide with a different set of mutations. Additionally, in some aspects, antibodies or antigen-biding fragments bind to a polypeptide described herein.

In certain embodiments, the compositions comprise a nucleic acid, such as an mRNA, that encodes the polypeptide. The mRNA, in some embodiments, has features that enable its successful use as a vaccine, such as a 5′ cap, 5′-untranslated region, a 3′-untranslated region, and a poly(A) tail. The mRNA, in some embodiments, comprises at least one non-canonical nucleobase (e.g., to improve its stability).

An mRNA comprising one or more non-canonical nucleosides or nucleotides, for example, is called a “modified” RNA to describe the presence of one or more non-naturally and/or naturally occurring components or configurations that are used instead of or in addition to the canonical A, G, C, and U residues.

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA. Certain embodiments comprise a 3′ end modification to an mRNA. A modified RNA can contain 5′ end and 3′ end modifications. A modified RNA can contain one or more modified residues at non-terminal locations. In certain embodiments, an mRNA includes at least one modified residue.

In some embodiments, the mRNA comprises SEQ ID NO: 2, which is provided below (with “T” shown instead of “U”).

>ENA|QHD43416|QHD43416.1 Severe acute respiratory syndrome
coronavirus 2 surface glycoprotein
ATGTTTGTTTTTCTTGTTTTATTGCCACTAGTCTCTAGTCAGTGTGTTAATCTTACAACC
AGAACTCAATTACCCCCTGCATACACTAATTCTTTCACACGTGGTGTTTATTACCCTGAC
AAAGTTTTCAGATCCTCAGTTTTACATTCAACTCAGGACTTGTTCTTACCTTTCTTTTCC
AATGTTACTTGGTTCCATGCTATACATGTCTCTGGGACCAATGGTACTAAGAGGTTTGAT
AACCCTGTCCTACCATTTAATGATGGTGTTTATTTTGCTTCCACTGAGAAGTCTAACATA
ATAAGAGGCTGGATTTTTGGTACTACTTTAGATTCGAAGACCCAGTCCCTACTTATTGTT
AATAACGCTACTAATGTTGTTATTAAAGTCTGTGAATTTCAATTTTGTAATGATCCATTT
TTGGGTGTTTATTACCACAAAAACAACAAAAGTTGGATGGAAAGTGAGTTCAGAGTTTAT
TCTAGTGCGAATAATTGCACTTTTGAATATGTCTCTCAGCCTTTTCTTATGGACCTTGAA
GGAAAACAGGGTAATTTCAAAAATCTTAGGGAATTTGTGTTTAAGAATATTGATGGTTAT
TTTAAAATATATTCTAAGCACACGCCTATTAATTTAGTGCGTGATCTCCCTCAGGGTTTT
TCGGCTTTAGAACCATTGGTAGATTTGCCAATAGGTATTAACATCACTAGGTTTCAAACT
TTACTTGCTTTACATAGAAGTTATTTGACTCCTGGTGATTCTTCTTCAGGTTGGACAGCT
GGTGCTGCAGCTTATTATGTGGGTTATCTTCAACCTAGGACTTTTCTATTAAAATATAAT
GAAAATGGAACCATTACAGATGCTGTAGACTGTGCACTTGACCCTCTCTCAGAAACAAAG
TGTACGTTGAAATCCTTCACTGTAGAAAAAGGAATCTATCAAACTTCTAACTTTAGAGTC
CAACCAACAGAATCTATTGTTAGATTTCCTAATATTACAAACTTGTGCCCTTTTGGTGAA
GTTTTTAACGCCACCAGATTTGCATCTGTTTATGCTTGGAACAGGAAGAGAATCAGCAAC
TGTGTTGCTGATTATTCTGTCCTATATAATTCCGCATCATTTTCCACTTTTAAGTGTTAT
GGAGTGTCTCCTACTAAATTAAATGATCTCTGCTTTACTAATGTCTATGCAGATTCATTT
GTAATTAGAGGTGATGAAGTCAGACAAATCGCTCCAGGGCAAACTGGAAAGATTGCTGAT
TATAATTATAAATTACCAGATGATTTTACAGGCTGCGTTATAGCTTGGAATTCTAACAAT
CTTGATTCTAAGGTTGGTGGTAATTATAATTACCTGTATAGATTGTTTAGGAAGTCTAAT
CTCAAACCTTTTGAGAGAGATATTTCAACTGAAATCTATCAGGCCGGTAGCACACCTTGT
AATGGTGTTGAAGGTTTTAATTGTTACTTTCCTTTACAATCATATGGTTTCCAACCCACT
AATGGTGTTGGTTACCAACCATACAGAGTAGTAGTACTTTCTTTTGAACTTCTACATGCA
CCAGCAACTGTTTGTGGACCTAAAAAGTCTACTAATTTGGTTAAAAACAAATGTGTCAAT
TTCAACTTCAATGGTTTAACAGGCACAGGTGTTCTTACTGAGTCTAACAAAAAGTTTCTG
CCTTTCCAACAATTTGGCAGAGACATTGCTGACACTACTGATGCTGTCCGTGATCCACAG
ACACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAGTC
CCTGTTGCTATTCATGCAGATCAACTTACTCCTACTTGGCGTGTTTATTCTACAGGTTCT
AATGTTTTTCAAACACGTGCAGGCTGTTTAATAGGGGCTGAACATGTCAACAACTCATAT
GAGTGTGACATACCCATTGGTGCAGGTATATGCGCTAGTTATCAGACTCAGACTAATTCT
CCTCGGCGGGCACGTAGTGTAGCTAGTCAATCCATCATTGCCTACACTATGTCACTTGGT
GCAGAAAATTCAGTTGCTTACTCTAATAACTCTATTGCCATACCCACAAATTTTACTATT
AGTGTTACCACAGAAATTCTACCAGTGTCTATGACCAAGACATCAGTAGATTGTACAATG
TACATTTGTGGTGATTCAACTGAATGCAGCAATCTTTTGTTGCAATATGGCAGTTTTTGT
ACACAATTAAACCGTGCTTTAACTGGAATAGCTGTTGAACAAGACAAAAACACCCAAGAA
GTTTTTGCACAAGTCAAACAAATTTACAAAACACCACCAATTAAAGATTTTGGTGGTTTT
AATTTTTCACAAATATTACCAGATCCATCAAAACCAAGCAAGAGGTCATTTATTGAAGAT
CTACTTTTCAACAAAGTGACACTTGCAGATGCTGGCTTCATCAAACAATATGGTGATTGC
CTTGGTGATATTGCTGCTAGAGACCTCATTTGTGCACAAAAGTTTAACGGCCTTACTGTT
TTGCCACCTTTGCTCACAGATGAAATGATTGCTCAATACACTTCTGCACTGTTAGCGGGT
ACAATCACTTCTGGTTGGACCTTTGGTGCAGGTGCTGCATTACAAATACCATTTGCTATG
CAAATGGCTTATAGGTTTAATGGTATTGGAGTTACACAGAATGTTCTCTATGAGAACCAA
AAATTGATTGCCAACCAATTTAATAGTGCTATTGGCAAAATTCAAGACTCACTTTCTTCC
ACAGCAAGTGCACTTGGAAAACTTCAAGATGTGGTCAACCAAAATGCACAAGCTTTAAAC
ACGCTTGTTAAACAACTTAGCTCCAATTTTGGTGCAATTTCAAGTGTTTTAAATGATATC
CTTTCACGTCTTGACAAAGTTGAGGCTGAAGTGCAAATTGATAGGTTGATCACAGGCAGA
CTTCAAAGTTTGCAGACATATGTGACTCAACAATTAATTAGAGCTGCAGAAATCAGAGCT
TCTGCTAATCTTGCTGCTACTAAAATGTCAGAGTGTGTACTTGGACAATCAAAAAGAGTT
GATTTTTGTGGAAAGGGCTATCATCTTATGTCCTTCCCTCAGTCAGCACCTCATGGTGTA
GTCTTCTTGCATGTGACTTATGTCCCTGCACAAGAAAAGAACTTCACAACTGCTCCTGCC
ATTTGTCATGATGGAAAAGCACACTTTCCTCGTGAAGGTGTCTTTGTTTCAAATGGCACA
CACTGGTTTGTAACACAAAGGAATTTTTATGAACCACAAATCATTACTACAGACAACACA
TTTGTGTCTGGTAACTGTGATGTTGTAATAGGAATTGTCAACAACACAGTTTATGATCCT
TTGCAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTCAA
AAAGAAATTGACCGCCTCAATGAGGTTGCCAAGAATTTAAATGAATCTCTCATCGATCTC
CAAGAACTTGGAAAGTATGAGCAGTATATAAAATGGCCATGGTACATTTGGCTAGGTTTT
ATAGCTGGCTTGATTGCCATAGTAATGGTGACAATTATGCTTTGCTGTATGACCAGTTGC
TGTAGTTGTCTCAAGGGCTGTTGTTCTTGTGGATCCTGCTGCAAATTTGATGAAGACGAC
TCTGAGCCAGTGCTCAAAGGAGTCAAATTACATTACACATAA

In some embodiments, the mRNA comprises a part of SEQ ID NO: 2, or a modified (e.g., codon-optimized) version of SEQ ID NO: 2 with the requisite mutations to encode the described polypeptides. In some embodiments, the modified version of the mRNA includes one or more (e.g., plurality, all) modified uridines. “Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, e.g., a pseudouridine in which one or more non-proton substituents (e.g., alkyl, such as methyl) takes the place of a proton. In some embodiments, a modified uridine is any of a substituted uridine, pseudouridine, or a substituted pseudouridine.

In some embodiments, the mRNA comprises at least one UTR from an expressed mammalian mRNA, such as a constitutively expressed mRNA. An mRNA is considered constitutively expressed in a mammal if it is continually transcribed in at least one tissue of a healthy adult mammal. In some embodiments, the mRNA comprises a 5′ UTR, 3′ UTR, or 5′ and 3′ UTRs from an expressed mammalian RNA, such as a constitutively expressed mammalian mRNA. Actin mRNA is an example of a constitutively expressed mRNA.

In some embodiments, the mRNA comprises at least one UTR from Hydroxysteroid 17-Beta Dehydrogenase 4 (HSD 17B4 or HSD), e.g., a 5′ UTR from HSD. In some embodiments, the mRNA comprises at least one UTR from a globin mRNA, for example, human alpha globin (HBA) mRNA, human beta globin (HBB) mRNA, or Xenopus laevis beta globin (XBG) mRNA. In some embodiments, the mRNA comprises a 5′ UTR, 3′ UTR, or 5′ and 3′ UTRs from a globin mRNA, such as HBA, HBB, or XBG. In some embodiments, the mRNA comprises a 5′ UTR from bovine growth hormone, cytomegalovirus (CMV), mouse Hba-a1, HSD, an albumin gene, HBA, HBB, or XBG. In some embodiments, the mRNA comprises a 3′ UTR from bovine growth hormone, cytomegalovirus, mouse Hba-a1, HSD, an albumin gene, HBA, HBB, or XBG. In some embodiments, the mRNA comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the mRNA comprises 5′ and 3′ UTRs that are from the same source, e.g., a constitutively expressed mRNA such as actin, albumin, or a globin such as HBA, HBB, or XBG.

In some embodiments, the mRNA does not comprise a 5′ UTR, e.g., there are no additional nucleotides between the 5′ cap and the start codon. In some embodiments, the mRNA comprises a Kozak sequence between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the mRNA comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase. In some embodiments, the Kozak sequence is rccAUGg with zero mismatches or with up to one or two mismatches to positions in lowercase. In some embodiments, the Kozak sequence is gccRccAUGG (SEQ ID NO: 3) with zero mismatches or with up to one, two, or three mismatches to positions in lowercase. In some embodiments, the Kozak sequence is gccAccAUG (SEQ ID NO: 4) with zero mismatches or with up to one, two, three, or four mismatches to positions in lowercase. In some embodiments, the Kozak sequence is GCCACCAUG. In some embodiments, the Kozak sequence is gccgccRccAUGG with zero mismatches or with up to one, two, three, or four mismatches to positions in lowercase.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33): 12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its DI subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines (SEQ ID NO: 5). In some embodiments, the poly-A tail comprises 95, 96, 97, 98, 99, or 100 adenine nucleotides (SEQ ID NO: 6). In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. As used herein, “non-adenine nucleotides” refer to any natural or non-natural nucleotides that do not comprise adenine. Guanine, thymine, and cytosine nucleotides are exemplary non-adenine nucleotides.

In some embodiments, the mRNA is purified. In some embodiments, the mRNA is purified using a precipitation method (e.g., LiCl precipitation, alcohol precipitation, or an equivalent method, e.g., as described herein). In some embodiments, the mRNA is purified using a chromatography-based method, such as an HPLC-based method or an equivalent method (e.g., as described herein). In some embodiments, the mRNA is purified using both a precipitation method (e.g., LiCl precipitation) and an HPLC-based method.

### Formulations

In some aspects, formulations comprise the polypeptides or nucleic acids described herein. The formulations, in some embodiments, further comprise at least one excipient. In some embodiments, the formulations further comprise a delivery system (e.g., selected from protamine, protamine liposome, polysaccharide particles, cationic nanoemulsion, cationic polymer, cationic polymer liposome, cationic lipid nanoparticle, cationic lipid/cholesterol nanoparticles, cationic lipid/cholesterol/PEG nanoparticles, and dendrimer nanoparticles). Various details of such formulations can be found in Pardi et al., mRNA vaccines—a new era in vaccinology, Nature Reviews—Drug Discovery 17: 261-279 (2018). The formulations, in some aspects, include a disclosed polypeptide and an adjuvant or a disclosed nucleic acid as part of a vector or transfection system.

For facilitating delivery of nucleic acids, such as mRNAs, certain lipid formulations can be used, as described further below.

In some embodiments, the lipid formulations, mRNA modifications, and other features of the formulations are as described in the following patents: U.S. Pat. Nos. 10,703,789; 10,702,600; 10,577,403; 10,442,756; 10,266,485; 10,064,959; 9,868,692, each of which is incorporated by reference in its entirety. In some embodiments, the formulations comprise lipids (SM-102, polyethylene glycol [PEG] 2000 dimyristoyl glycerol [DMG], cholesterol, and 1,2-distearoyl-sn-glycero-3-phosphocholine [DSPC]), tromethamine, tromethamine hydrochloride, acetic acid, sodium acetate trihydrate, and/or sucrose.

Disclosed herein are various embodiments of LNP formulations for biologically active agents, such as RNAs. Such LNP formulations include an “amine lipid” or a “biodegradable lipid”, optionally along with one or more of a helper lipid, a neutral lipid, and a stealth lipid such as a PEG lipid. By “lipid nanoparticle” is meant a particle that comprises a plurality of (i.e. more than one) lipid molecules physically associated with each other by intermolecular forces.

In certain embodiments, LNP compositions for the delivery of biologically active agents comprise an “amine lipid”, which is defined as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Lipid A may be synthesized according to WO2015/095340 (e.g., pp. 84-86). In certain embodiments, the amine lipid is an equivalent to Lipid A.

In certain embodiments, an amine lipid is an analog of Lipid A. In certain embodiments, a Lipid A analog is an acetal analog of Lipid A. In particular LNP compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids and other “biodegradable lipids” suitable for use in the LNPs described herein are biodegradable in vivo. The amine lipids have low toxicity (e.g., are tolerated in animal models without adverse effect in amounts of greater than or equal to 10 mg/kg). In certain embodiments, LNPs comprising an amine lipid include those where at least 75% of the amine lipid is cleared from the plasma within 8, 10, 12, 24, or 48 hours, or 3, 4, 5, 6, 7, or 10 days. In certain embodiments. LNPs comprising an amine lipid include those where at least 50% of the mRNA is cleared from the plasma within 8, 10, 12, 24, or 48 hours, or 3, 4, 5, 6, 7, or 10 days. In certain embodiments, LNPs comprising an amine lipid include those where at least 50% of the LNP is cleared from the plasma within 8, 10, 12, 24, or 48 hours, or 3, 4, 5, 6, 7, or 10 days, for example by measuring a lipid (e.g. an amine lipid), RNA (e.g. mRNA), or other component. In certain embodiments, lipid-encapsulated versus free lipid, RNA, or nucleic acid component of the LNP is measured.

Biodegradable lipids include, for example the biodegradable lipids of WO/2017/173054, WO2015/095340, and WO2014/136086. Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”).

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or assymetric.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g, J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15.000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide. PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Ala., USA), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

The LNP may contain (i) a biodegradable lipid, (ii) an optional neutral lipid, (iii) a helper lipid, and (iv) a stealth lipid, such as a PEG lipid. The LNP may contain a biodegradable lipid and one or more of a neutral lipid, a helper lipid, and a stealth lipid, such as a PEG lipid.

The LNP may contain (i) an amine lipid for encapsulation and for endosomal escape, (ii) a neutral lipid for stabilization, (iii) a helper lipid, also for stabilization, and (iv) a stealth lipid, such as a PEG lipid. The LNP may contain an amine lipid and one or more of a neutral lipid, a helper lipid, also for stabilization, and a stealth lipid, such as a PEG lipid.

In certain embodiments, lipid compositions are described according to the respective molar ratios of the component lipids in the formulation. Embodiments of the present disclosure provide lipid compositions described according to the respective molar ratios of the component lipids in the formulation. In one embodiment, the mol-% of the amine lipid may be from about 30 mol-% to about 60 mol-%. In one embodiment, the mol-% of the amine lipid may be from about 40 mol-% to about 60 mol-%. In one embodiment, the mol-% of the amine lipid may be from about 45 mol-% to about 60 mol-%. In one embodiment, the mol-% of the amine lipid may be from about 50 mol-% to about 60 mol-%. In one embodiment, the mol-% of the amine lipid may be from about 55 mol-% to about 60 mol-%. In one embodiment, the mol-% of the amine lipid may be from about 50 mol-% to about 55 mol-%. In one embodiment, the mol-% of the amine lipid may be about 50 mol-%. In one embodiment, the mol-% of the amine lipid may be about 55 mol-%. In some embodiments, the amine lipid mol-% of the LNP batch will be ±30%, ±25%, ±20%, ±15%, ±10%, ±5%, or ±2.5% of the target mol-%. In some embodiments, the amine lipid mol-% of the LNP batch will be ±4 mol-%, ±3 mol-%, ±2 mol-%, ±1.5 mol-%, ±1 mol-%, ±0.5 mol-%, or ±0.25 mol-% of the target mol-%. All mol-% numbers are given as a fraction of the lipid component of the LNP compositions. In certain embodiments, LNP inter-lot variability of the amine lipid mol-% will be less than 15%, less than 10% or less than 5%.

In one embodiment, the mol-% of the neutral lipid may be from about 5 mol-% to about 15 mol-%. In one embodiment, the mol-% of the neutral lipid may be from about 7 mol-% to about 12 mol-%. In one embodiment, the mol-% of the neutral lipid may be about 9 mol-%. In some embodiments, the neutral lipid mol-% of the LNP batch will be ±30%, ±25%, ±20%, ±15%, ±10%, ±5%, or ±2.5% of the target neutral lipid mol-%. In certain embodiments, LNP inter-lot variability will be less than 15%, less than 10% or less than 5%.

In one embodiment, the mol-% of the helper lipid may be from about 20 mol-% to about 60 mol-%. In one embodiment, the mol-% of the helper lipid may be from about 25 mol-% to about 55 mol-%. In one embodiment, the mol-% of the helper lipid may be from about 25 mol-% to about 50 mol-%. In one embodiment, the mol-% of the helper lipid may be from about 25 mol-% to about 40 mol-%. In one embodiment, the mol-% of the helper lipid may be from about 30 mol-% to about 50 mol-%. In one embodiment, the mol-% of the helper lipid may be from about 30 mol-% to about 40 mol-%. In one embodiment, the mol-% of the helper lipid is adjusted based on amine lipid, neutral lipid, and PEG lipid concentrations to bring the lipid component to 100 mol-%. In some embodiments, the helper mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

In one embodiment, the mol-% of the PEG lipid may be from about 1 mol-% to about 10 mol-%. In one embodiment, the mol-% of the PEG lipid may be from about 2 mol-% to about 10 mol-%. In one embodiment, the mol-% of the PEG lipid may be from about 2 mol-% to about 8 mol-%. In one embodiment, the mol-% of the PEG lipid may be from about 2 mol-% to about 4 mol-%. In one embodiment, the mol-% of the PEG lipid may be from about 2.5 mol-% to about 4 mol-%. In one embodiment, the mol-% of the PEG lipid may be about 3 mol-%. In one embodiment, the mol-% of the PEG lipid may be about 2.5 mol-%. In some embodiments, the PEG lipid mol-% of the LNP batch will be ±30%, ±25%, ±20%, ±15%, ±10%, ±5%, or ±2.5% of the target PEG lipid mol-%. In certain embodiments, LNP inter-lot variability will be less than 15%, less than 10% or less than 5%.

In certain embodiments, the cargo includes an mRNA encoding one or more of the disclosed polypeptides. In one embodiment, an LNP composition may comprise a Lipid A or its equivalents. In some aspects, the amine lipid is Lipid A. In some aspects, the amine lipid is a Lipid A equivalent, e.g. an analog of Lipid A. In certain aspects, the amine lipid is an acetal analog of Lipid A. In various embodiments, an LNP composition comprises an amine lipid, a neutral lipid, a helper lipid, and a PEG lipid. In certain embodiments, the helper lipid is cholesterol. In certain embodiments, the neutral lipid is DSPC. In specific embodiments, PEG lipid is PEG2k-DMG. In some embodiments, an LNP composition may comprise a Lipid A, a helper lipid, a neutral lipid, and a PEG lipid. In some embodiments, an LNP composition comprises an amine lipid, DSPC, cholesterol, and a PEG lipid. In some embodiments, the LNP composition comprises a PEG lipid comprising DMG. In certain embodiments, the amine lipid is selected from Lipid A, and an equivalent of Lipid A, including an acetal analog of Lipid A. In additional embodiments, an LNP composition comprises Lipid A, cholesterol, DSPC, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, an LNP composition may comprise a lipid component that comprises an amine lipid, a helper lipid, a neutral lipid, and a helper lipid; and a nucleic acid component, wherein the N/P ratio is about 3 to 10. In some embodiments, an LNP composition may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be ±30%, ±25%, ±20%, ±15%, ±10%, ±5%, or ±2.5% of the target N/P ratio. In certain embodiments, LNP inter-lot variability will be less than 15%, less than 10% or less than 5%.

In some embodiments, LNPs are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of LNPs, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt. buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, tangential flow filtration, or chromatography. The LNPs may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at 4° C., at room temperature, or at 25° C. Frozen LNP compositions may be maintained at various temperatures, for example on ice, at 4° C., at room temperature, at 25° C., or at 37° C.

### Methods Related to the Polypeptides, Mutations, and Formulations

In some aspects, the methods are disclosed that use the described polypeptides, nucleic acids, compositions, or formulations.

For example, methods of vaccinating a subject against SARS-CoV-2 infection comprise administering to the subject a composition or a formulation according to any of the described embodiments. The administering step, in some embodiments, is via intramuscular injection or intradermal injection.

In some aspects, methods of selecting an antibody for treating a SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a SARS-CoV-2 spike protein from the subject; and selecting an antibody that does not bind to the N-terminal domain antigenic supersite of the SARS-CoV-2 spike protein.

In some aspects, methods of selecting a convalescent plasma against SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a SARS-CoV-2 spike protein from the subject; and selecting a convalescent plasma having antibodies that do not bind to the N-terminal domain antigenic supersite of the SARS-CoV-2 spike protein.

In some aspects, methods of selecting a vaccine against SARS-CoV-2 infection in a subject comprise determining the presence of one or more mutations at a residue selected from 14-16, 24-26, 63-76, 81, 85-89, 136-146, 150-165, 167-169, 210-212, 214, 216 and 241-252 in a spike protein from an emerging variant of SARS-CoV-2; and selecting a vaccine having a polypeptide according to any of the embodiments described herein.

### Example

### SARS-CoV-2 Antigenic Minimalism Linked to Surges in Community Transmission and Vaccine Breakthrough Infections

The raging COVID-19 pandemic in India, combined with cases of re-infection and post-vaccination “breakthrough” globally have raised alarm, mandating characterization of the immuno-evasive features of SARS-CoV-2. Here, we systematically analyzed over 1.3 million SARS-CoV-2 genomes from 178 countries and also conducted whole-genome viral sequencing from 53 patients at Mayo Clinic sites that had developed SARS-CoV-2 re-infections or vaccine breakthrough infections. We identified 116 Spike protein mutations that increased in prevalence during at least one surge in PCR test positivity in any country over a three-month window. Deletions in the Spike protein N-terminal domain (NTD) are enriched for these ‘surge-associated mutations’ (Odds Ratio=1.96, 95% CI: 1.35-2.85; p<0.001) and are expanding into longer contiguous stretches of deletions over the course of the pandemic. In the ongoing COVID-19 surge in India, an emerging NTD deletion (ΔF157/R158) has increased over 10-fold in prevalence from February 2021 (1.1%) to April 2021 (15%). During the recent surge in Chile, a hitherto uncharacterized NTD deletion (Δ246-252) has increased in prevalence by over 30-fold from January 2021 (0.86%) to April 2021 (33%). Strikingly, these emerging surge-associated deletions in India and Chile map directly to an antigenic supersite that is bound by most NTD-targeted neutralizing antibodies. Finally, in three patients from Mayo Clinic in Minnesota who were previously infected or vaccinated, we identified NTD deletions (Δ85-90, Δ156-164, Δ167-174) that were never previously found in the state. These putative immune escape deletions are also proximal to the neutralizing antibody binding sites, suggesting that antigenic minimalism may be an emerging evolutionary strategy for SARS-CoV-2 to evade immune responses. This study highlights the urgent need to sequence SARS-CoV-2 genomes at much larger scale globally and mandate a public health policy for more granular and transparent reporting of SARS-CoV-2 sample annotations such as de-identified patient phenotypes and vaccination status. Such a universal standard for genomic epidemiology and clinical genomics is imperative to proactively predict breakthrough and reinfection mutations at their incipient stages, as well as guide the development of neutralizing antibodies and future COVID-19 vaccines that thwart a broad spectrum of immunoevasive SARS-CoV-2 variants.

### Introduction

The ongoing COVID-19 pandemic has infected around 500 million people and killed more than 6.1 million people worldwide, as of April 2022¹. The continual emergence of SARS-CoV-2 variants with increased transmissibility and capacity for immune escape, such as B.1.17 (“UK variant”) and P.1 (“Brazilian variant”), threatens to prolong the pandemic through devastating outbreaks such as the one currently being witnessed in India². While multiple vaccines have demonstrated high effectiveness in clinical trials and real world studies³⁻⁵, there have been reports of “vaccine breakthrough infections” with SARS-CoV-2 variants^{6,7}. A recent study described two such cases in New York, at least one of which occurred despite confirmation of a robust neutralizing antibody response. Variant classification schemes have been developed by the US Centers for Disease Control and Prevention (CDC)⁸ and the World Health Organisation (WHO)⁹ based on factors such as prevalence, evidence of transmissibility and disease severity, and ability to be neutralized by existing therapeutics or sera from vaccinated patients. Early and rapid detection of these emerging Variants of Concern/Interest is imperative to combat and contain the ongoing pandemic and future outbreaks.

It is critical to thoroughly characterize how SARS-CoV-2 mutates to evade natural and vaccine-induced immune responses as it continues driving case surges. To this end, neutralizing antibodies which target the receptor-binding domain (RBD) or the N-terminal domain (NTD) of the Spike protein have been isolated from the sera of COVID-19 patients¹⁰⁻¹². Recent studies contemporaneously found that several neutralizing antibodies target a single antigenic supersite in the NTD of the Spike protein^{13,14}. The NTD is also a hotspot for in-frame deletions in the SARS-CoV-2 genome, with four recurrent deletion regions (RDRs) identified¹⁵. Several such deletions have been experimentally demonstrated to reduce neutralization by some NTD-targeting neutralizing antibodies^{13,15}. Whether additional deletions have emerged in variants that drive surges or vaccine breakthrough infections needs to be determined.

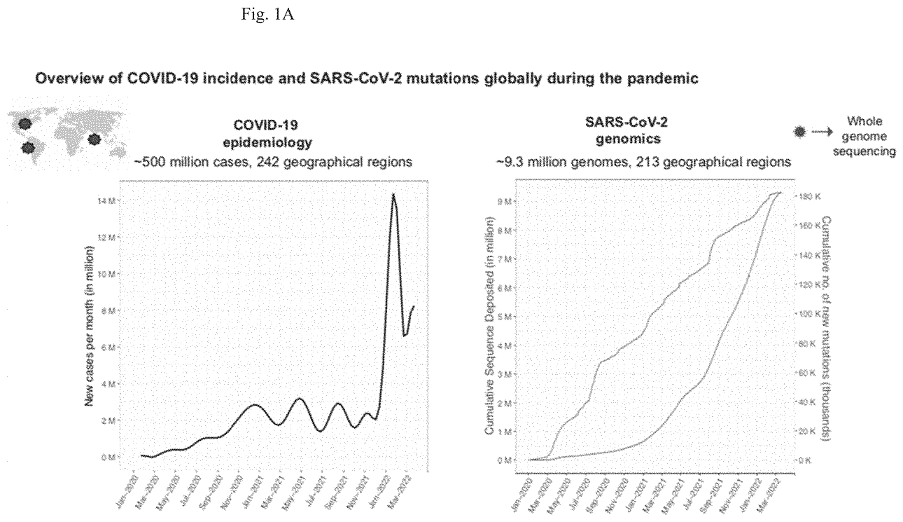

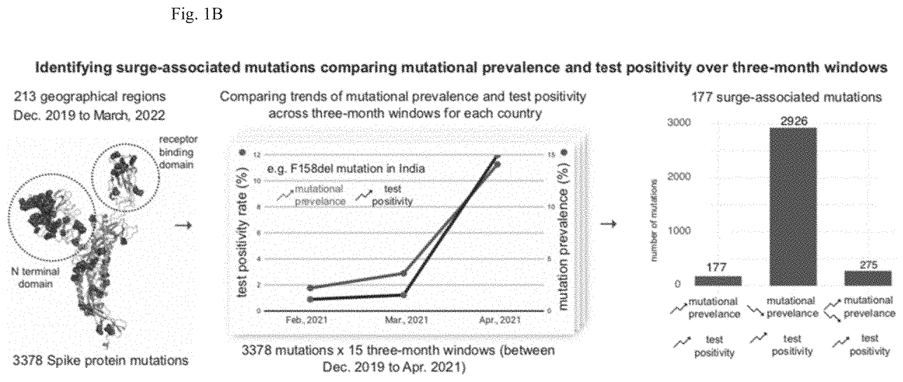

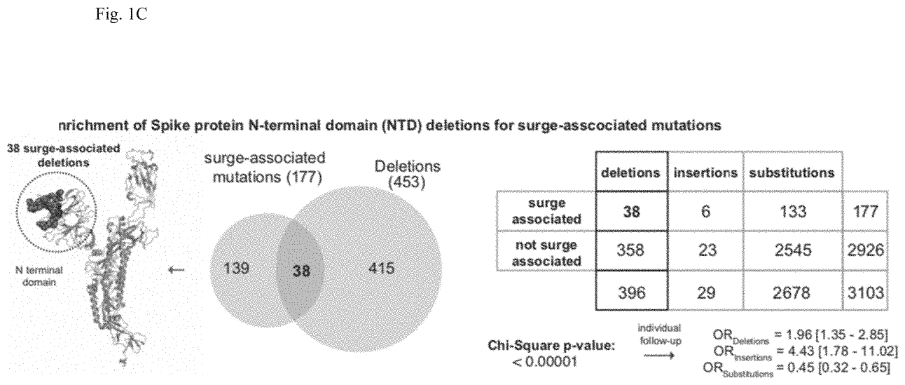

Concerted global data sharing efforts during the pandemic have led to the rapid development of large-scale genomic and epidemiological COVID-19 resources. Over 9.3 million SARS-CoV-2 genomes from 213 distinct geographical regions have been deposited throughout the pandemic in the GISAID database (FIG. 1). In addition, we are generating whole-genome viral sequences of SARS-CoV-2 from patients at the Mayo Clinic that had developed SARS-CoV-2 re-infections or post-vaccination “breakthrough” infections. On the epidemiology front, population-level metrics including SARS-CoV-2 positivity rates and mortality rates are being collected from 219 countries in databases such as OWID. The unprecedented availability of genomic-epidemiology data combined with clinical genomic data provides a timely opportunity to systematically characterize the immune evasive features of SARS-CoV-2.

In this study, we reveal that deletion mutations in the Spike protein have a high likelihood of being associated with surges in community transmission. We identify rapidly emerging surge-associated deletion mutations in India and Chile that map to a proposed antigenic supersite. We also identify non-overlapping deletion mutations in SARS-CoV-2 from patients with re-infection/vaccine-breakthrough infections, also mapping near the antibody-binding site and thus representing candidates for vaccine escape mutations. Finally, we highlight that the deletion-prone regions of the Spike protein are expanding during the course of the pandemic as an evolutionary strategy of “antigenic minimalism” to evade immune responses.

### Results

Deletions are Enriched for Association with Surges in Community Transmission of SARS-CoV-2

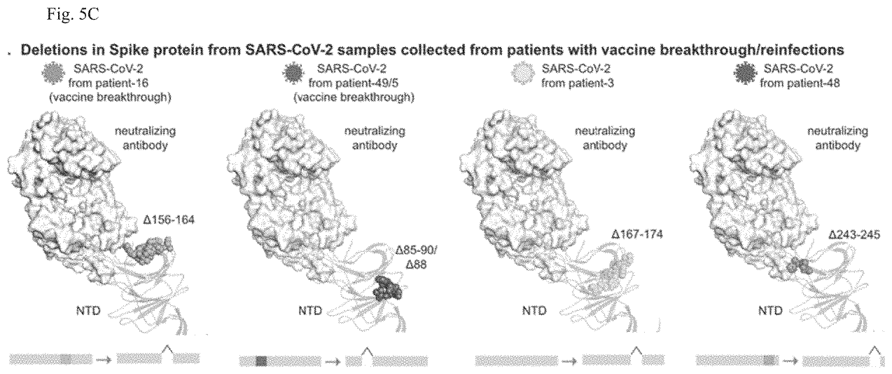

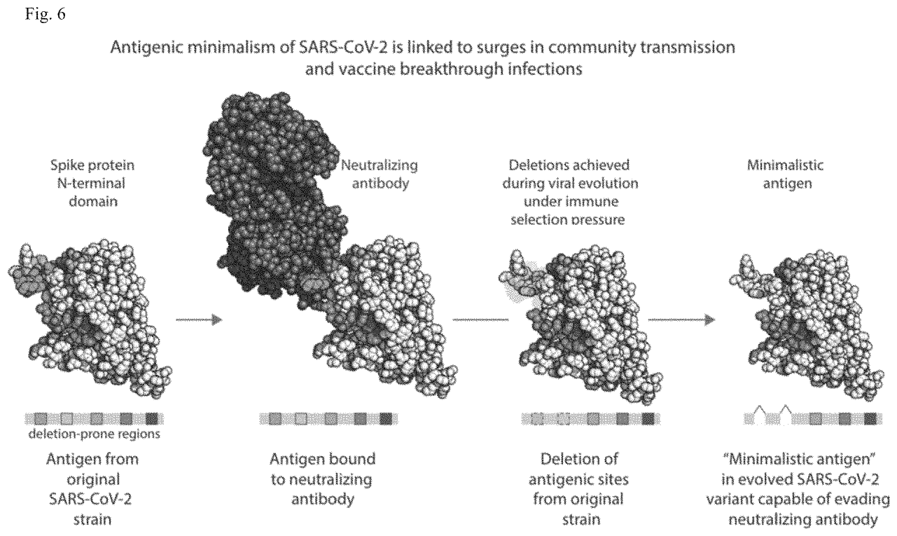

Analysis of 9,299,506 SARS-CoV-2 genome sequences (FIG. 1A) revealed the presence of 3410 amino acid mutations (missense and indels) in the Spike protein, spanning 85.86% of its residues (1093 out of 1273 residues). It is to be noted that these mutations were observed in 100 or more SARS-CoV-2 genome sequences, ensuring that these changes are not random occurrences from sequencing errors. These mutations include 2906 substitutions (95.7%), 453 deletions (4%), and 51 insertions (0.3%). To identify the mutations associated with surges in the community spread of COVID-19 (“surge-associated mutations”) during the pandemic, we sought to identify mutations that increased monotonically during periods of monotonically increasing test positivity (FIG. 1B). We identified 116 mutations that increased in prevalence during one or more surges in test positivity, in any country, over a three-month time interval. This approach recapitulated 45 out of 56 (80%) mutations known to be present in the CDC variants of interest or concern, including E484K, N501Y, D614G, P681H, P681R, ΔH69/V70, and ΔY144 (FIG. 6).

Further, we investigated whether a class of mutations (missense and/or indels) are enriched among the surge-associated mutations. 38 of 396 (9.5%) deletions were surge-associated, as compared to 133 of 2545 (5.22%) substitutions, and 6 of 29 (20.68%) insertions. This data indicates that deletions, but not substitutions or insertions, are enriched for association with surges (Chi-square Test p-value <0.00001; Odds Ratio=1.96, 95% CI: 1.35-2.85; FIG. 1C). The surge-associated deletions occur exclusively in the N-terminal domain (NTD), which is interesting in light of the fact that four immunogenicity-altering recurrent deletion regions (RDRs) in the NTD were recently identified the predominant sites of deletion in the Spike protein¹⁵. This suggests that genomic deletions may be an important immune evasion strategy for SARS-CoV-2 and can contribute to the community transmission of COVID-19.

Rapidly Emerging Deletion Mutations Associated with Surges in India and Chile Map to Antigenic Supersite Binding Most NTD-Targeted Neutralizing Antibodies

Recently there have been massive surges of COVID-19 infection in a few countries, most prominently in India¹⁶ and Chile^{17,18}. In order to identify the mutations associated with recent surges, we identified the mutations which have monotonically increased in frequency during a monotonic increase in test positivity in any country between February and April 2021. We found that different sets of mutations have increased in prevalence during current surges in seven countries: Poland, Bangladesh, Belgium, Chile, France, India and Sweden (Table 1).

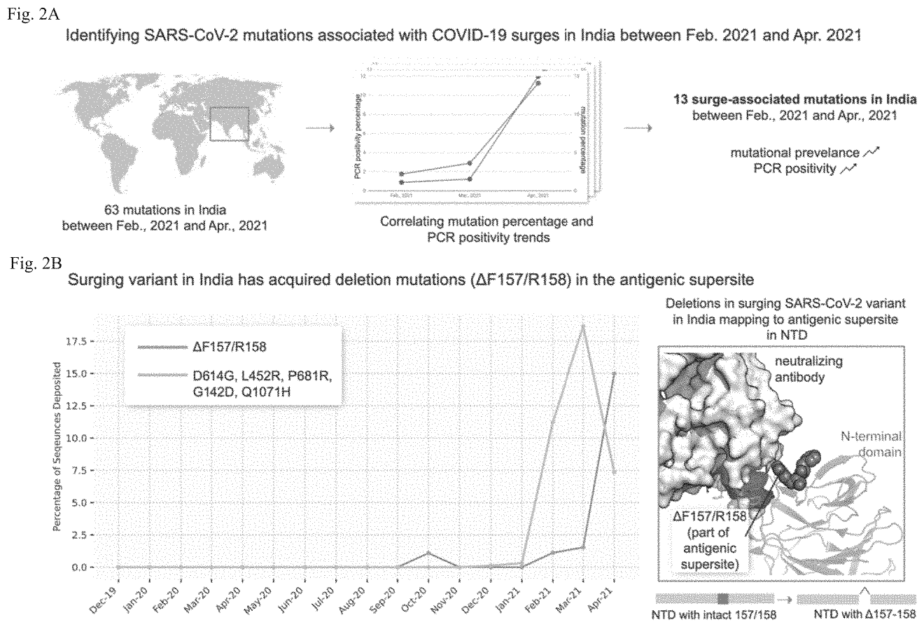

In India, 13 mutations are correlated with the recent massive surge (“second wave of infections”, in the month of April 2021), which includes an emerging deletion (ΔF157/R158) in the NTD. This deletion has co-occurred with the existing mutations (P681R, L452R, E484Q) and is found in B.1.617.2, which has been categorized as a variant of interest by the CDC⁸ (FIG. 2A, Table 4). There was a 13.6 fold increase in the prevalence of the ΔF157/R158 between February and April 2021, from 1.1% (of 1254 sequences) to 15% (of 367 sequences). Correspondingly, the test positivity rose from 1.8% in February 2021 to 11.3% in April 2021. Mapping the ΔF157/R158 region onto previously determined Spike-protein:neutralizing-antibody complex structures shows that F157 and R158 reside in the antigenic supersite, which is recognized by a number of NTD-targeting neutralizing antibodies^{14,19} (FIG. 2B). Importantly, this deletion had not been identified at the time of the prior characterization of Spike protein deletions, and thus we suggest that this may represent a novel distinct fifth RDR¹⁵. Based on the trends observed at other RDRs, we hypothesize that longer stretches of deletions will emerge in this region during the coming months.

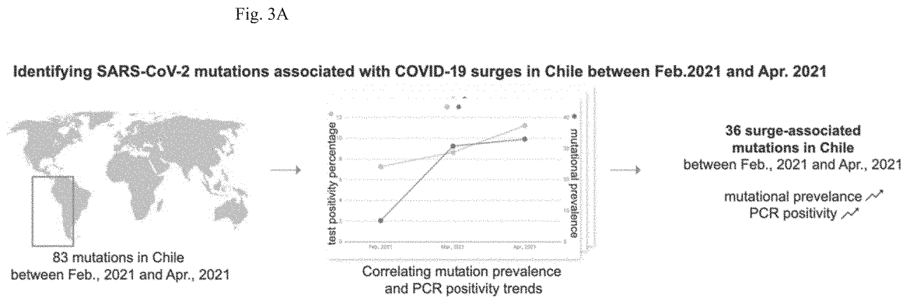

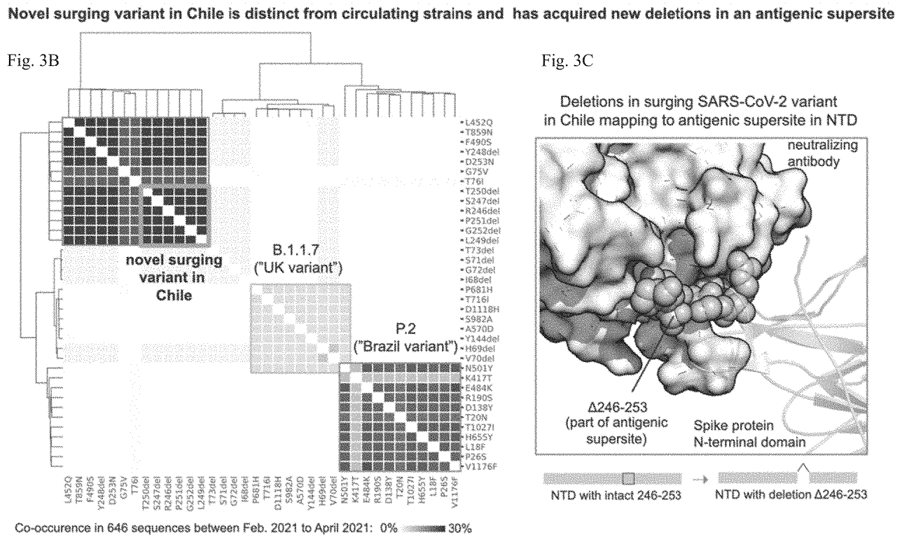

In Chile, 36 mutations are correlated with the current surge (April 2021), which clusters into three distinct groups corresponding to independently circulating variants (FIG. 3A, Table 5). One cluster includes mutations that are present in the UK variant (B.1.1.7): ΔH69/V70, ΔY144, A570D, P681H, T716I, S982A, D1118H. Another cluster has mutations overlapping with the Brazilian variant (P.2): L18F, T20N, P26S, D138Y, R1905, K417T, E484K, N501Y, D614G, H655Y, T1027I. Interestingly, the third emerging variant contains a deletion stretch (Δ246-252) in the NTD abutting, but not included in, a previously described recurrent region deletion (RDR4: Δ242-248)¹⁵. The Δ247-252 contiguous deletion has increased monotonically in frequency by over 30-fold from January to April 2020 (0.86% to 33.0%), during which time the test positivity has increased from 7.2% to 11.2%. ΔY248 was first observed in the United States in December 2020, albeit at a very low frequency (0.01%). Δ249-252 appears to have emerged later, with the earliest detection in Peru in February 2021. Interestingly, a structural analysis indicates that this region, like F157/R158, resides within the antigenic supersite (FIG. 3B) (Chi et al. 2020; Cerutti et al. 2021). Mapping this region onto the Spike-protein:neutralizing-antibody complex structure¹⁹, like F157/R158, we find that the 246-252 region also forms an epitope recognized by both T-cells and B-cells.

Taken together, this analysis highlights two NTD deletions that are rapidly emerging in specific countries and are strongly correlated with the surges in community spreads of SARS-CoV-2 in each. Furthermore, structures show that these residues are found in the binding sites for several characterized neutralizing antibodies. Deletion of these epitopes from the Spike protein is highly likely to diminish antibody binding affinity thereby enabling immune escape.

Analysis of SARS-CoV-2 Genomes from COVID-19 Patients with Vaccine Breakthrough Reveals the Presence of Distinct Deletions in the N-Terminal Domain

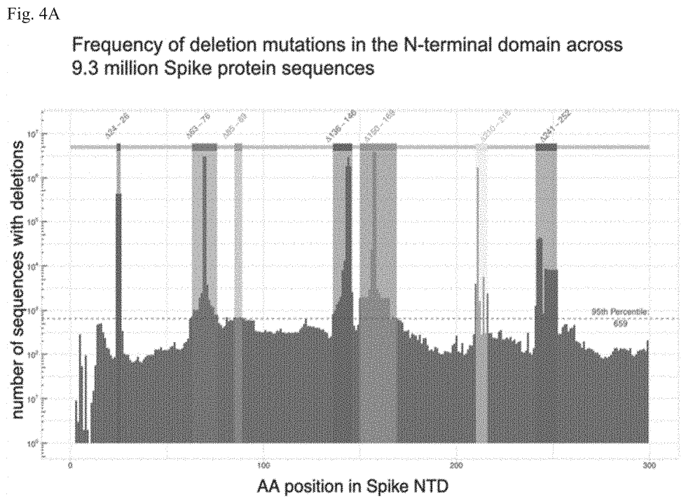

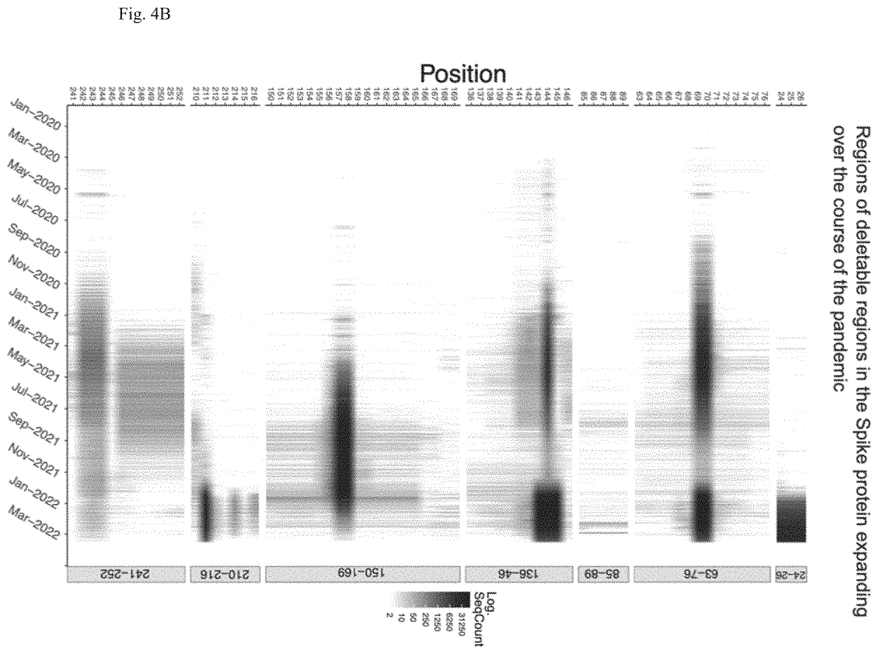

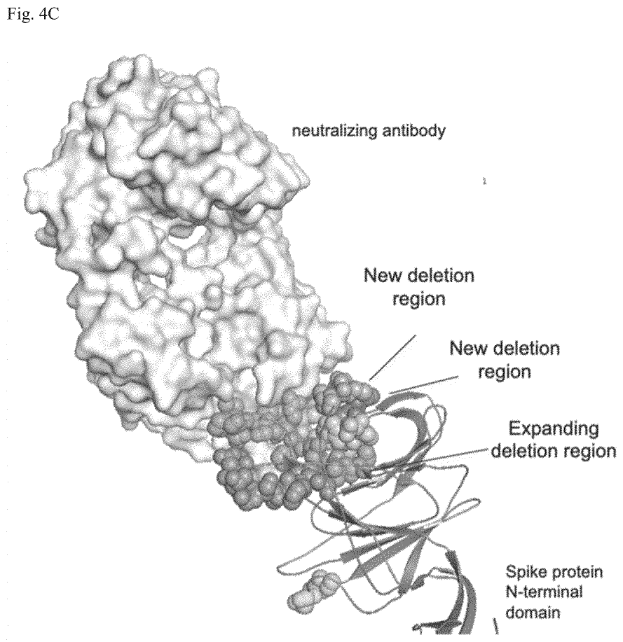

While the polyclonal nature of the immune response to vaccination makes it unlikely that single mutations will alter vaccine effectiveness, combinations of mutations may indeed lower the sensitivity of particular variants to vaccine-induced immunity. As such, it is important to track the sets of mutations that are present in variants infecting vaccinated individuals. To do so, we performed whole genome viral sequencing from 52 breakthrough COVID-19 cases in the Mayo Clinic health system. In total, we have identified 92 unique mutations, of which 29 are deletions (FIG. 4A). As expected, all observed Spike protein deletions in this cohort occurred in the NTD, with Δ144 and ΔH69/V70 showing the highest prevalence (64% and 62%, respectively).

We identified four variants harboring one or more less characterized deletion stretches. Importantly, each one had deletions in a distinct NTD region, demonstrating the genomic heterogeneity of vaccine escape variants and emphasizing that these cases of vaccine escape are not explained simply by the spread of one immuno-evasive strain of SARS-CoV-2. Whether the deletions were already present at the times of infection or evolved within these individuals under the pressure of vaccine-induced immunity is not known.

One patient who had received two doses of BTN162b2 in January 2021 was subsequently infected in April. The virus recovered from this patient contained a Δ156-164 deletion, reminiscent of the ΔF157/R158 which has increased in prevalence during the case surge in India (FIG. 4B). In another breakthrough infection, a patient who had the second dose of BTN162b2 vaccine in the beginning of April 2021, was subsequently observed to be reinfected by the end of April 2021. The sequence of SARS-CoV-2 virus recovered from this patient harboured a ΔD88 deletion in addition to ΔH69/V70 and ΔY144.

More interestingly, viral genomes recovered from two breakthrough cases contained deletions outside of the RDRs which we and others have identified from GISAID data¹⁵. One patient who was fully vaccinated with BNT162b2 in February was infected in March, and the recovered virus contained a Δ167-174 deletion (FIG. 4B). In another individual who was infected after one dose of BNT162b2, the virus harbored a Δ85-90 deletion (FIG. 4B). Only 867 of the 9.3 million deposited SARS-CoV-2 sequences in GSAID possessed a deletion of one or more amino acids between residues 85-90, 128 of these are from the United States (Table 3).

From a structural standpoint, all four deletions map to parts of the NTD that are either at the antigenic supersite or are proximal to it, as seen on the structure. The deletion of these loops is likely to result in lowered antibody binding and thereby may enable immune escape. Some residues reside in a flexible loop (FIG. 4C), which may indeed be more susceptible to acquiring mutations in the context of antigenic site minimalism. Overall, these observations raise the question whether the antibodies stimulated by BNT162b2 are effective against these deletion variants.

### Recurrent Deletion Regions in the Spike Protein can Emerge and Expand Over Time

The identification of deletion stretches outside of the four previously defined RDRs during test positivity surges (ΔF157/R158 in India) and in breakthrough infections (Δ85-90 and Δ167-174 in breakthrough cases at the Mayo Clinic) emphasizes that we must continue to vigilantly monitor deletion patterns to capture new RDRs as they emerge. Indeed, while the SARS-CoV-2 RDRs were initially defined based on 146,795 sequences deposited in GISAID as of Oct. 24, 2020, the number of deposited sequences has increased almost 10-fold over the past seven months.

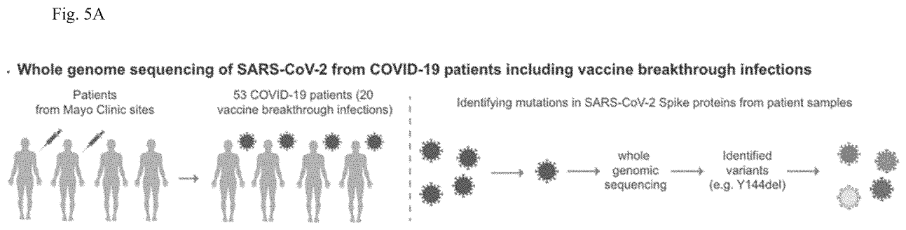

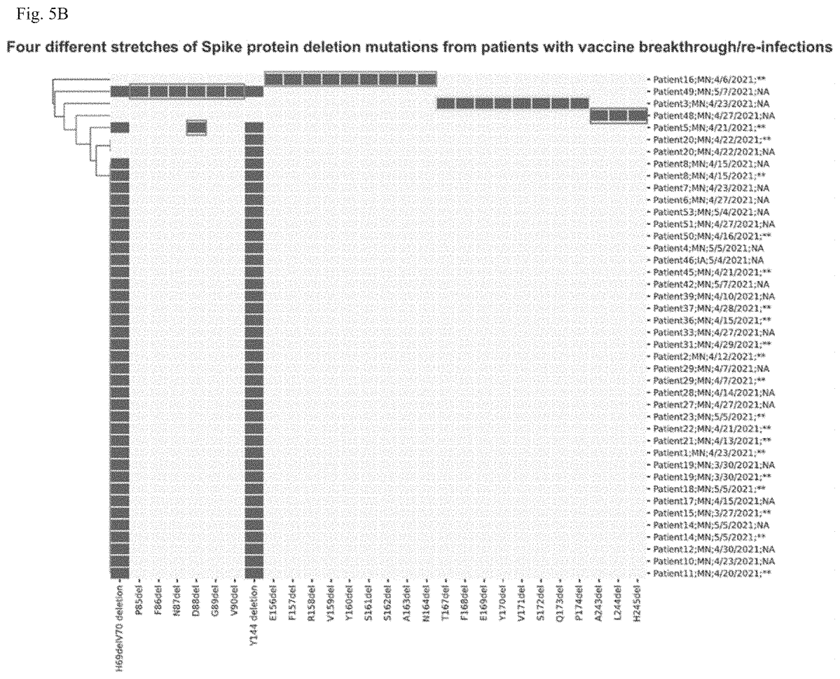

As such, we examined the current distribution of deletion frequencies for all amino acids in the Spike protein sequence to identify any additional candidate RDRs (FIG. 5A; see Methods). It should be noted in this context that all known deletions in the Spike protein sequence exclusively localize to the NTD. In addition to ΔF157/R158, we found that residues 14-16 (QCV) are deleted more frequently than expected based on the background distribution. Interestingly, these residues map to the same antigenic supersite as the other regions described previously (FIG. 5A). We confirmed that most viral genomes containing one or more deletions in this region were deposited after Oct. 24, 2020, explaining why this stretch was not captured in the initial characterization of RDRs¹⁵. We also identified potential RDRs at residues 5640/N641 and 675-681 (QTQTNSP (SEQ ID NO: 7)), the latter of which directly precedes the Spike protein furin cleavage site that we and others have described previously²⁰⁻²³. It is notable that these are the only RDRs observed to date that are outside of the NTD (and thus outside of the antigenic supersite), and their functional significance warrants follow-up.

In addition to identifying new RDRs, we also recognized that some RDRs appear to have the capacity to expand (i.e., to involve more flanking amino acids) over time. For example, the Δ246-252 deletion in one of the surge associated Chile variants can be viewed as an expansion of the previously defined RDR4 (Δ242-248)¹⁵ (FIG. 5B). Similarly, while we proposed that ΔF157/R158 (associated with the current surge in India) should be considered as a novel fifth RDR, our subsequent identification of Δ156-164 in a breakthrough infection suggests that this fifth RDR should actually be more defined with a wider sequence.

Taken together, our analysis highlights both the emergence of novel RDRs and the expansion of previously defined RDRs over the past several months. Given the clear need for dynamic classification, we suggest that nomenclature should henceforth be defined by residue numbers rather than sequential 5′ to 3′ order to avoid confusion when new RDRs arise which fall between two that have been previously characterized. As such, the currently existing RDRs in the NTD of the Spike protein can be defined as RDR14-16 (new RDR), RDR67-74 (part of previous RDR1), RDR138-146 (extended RDR2), RDR157-158 (new RDR), RDR210-211 (previous RDR3), and RDR241-252 (extended RDR4). Further, while they have not yet emerged to frequencies warranting an RDR classification in GISAID, the other regions with breakthrough infection-associated deletions (Δ85-90 and Δ167-174) should be monitored as candidates for emerging RDRs in the coming months. Our data suggests that experiments should be conducted to determine whether deletions in several NTD regions (residues 85-90, 156-159, 167-174, and 249-252) impact the binding of NTD-targeted neutralizing antibodies or the capacity of sera from vaccinated individuals to neutralize the virus.

### Discussion

The worldwide mass vaccination campaign has had a profound impact on COVID-19 transmission. However, certain variants are less susceptible to neutralization by sera from vaccinated individuals and convalescent COVID-19 patients^{24,25}. Such findings motivate the need to vigilantly track the emergence of new variants and to determine whether they are likely to cause surges or vaccine breakthrough infections. Here, through an integrated analysis of genomic and epidemiologic data, we found that deletions in the Spike protein NTD which map to an antigenic supersite have emerged over the course of the pandemic, are strongly associated with case surges, and are present in a subset of vaccine breakthrough variants. Indeed, in addition to deletion mutations several substitution mutations (e.g. E484Q, T478K in the receptor binding domain) are also associated with surges in cases (FIG. 1). Thus, a concerted evolution of strategically placed deletions and substitutions appear to be conferring SARS-CoV-2 with the fitness to evade immunity and achieve efficient transmission between hosts. Our finding that Spike protein NTD deletions are strongly enriched for association with test positivity surges is notable in the context of a previous report identifying the NTD as the most common site of deletions¹⁵. Specifically, this prior study highlighted four recurrent deletion regions in the NTD based on the GISAID data deposited as of October 2020 (146,795 total sequences). Several of these regions overlap with the putative residues of the recently identified NTD antigenic supersite, and deletions within them can abrogate binding to neutralizing antibodies¹³⁻¹⁵. Our study builds upon this prior work by examining the deletions which have arisen in the interim, during which over 1.1 million additional sequences have been deposited. In addition to validating the previously suggested definitions of RDR1 (ΔH69/V70 and flanking deletions), RDR2 (ΔY144 and flanking deletions), and RDR3 (ΔI210 and ΔN211), we found that RDR4 (previously defined as positions 242-248) has recently expanded to include positions 249-252. These residues are indeed part of the structurally mapped supersite^{13,14}, and a variant with the Δ248-252 deletion increased in prevalence during a recent test positivity surge in Chile. The recently evolved ΔF157/R158 deletion, which has expanded during the massive surge in India, marks a new RDR which also maps to the supersite¹⁴. Finally, our real time surveillance of SARS-CoV-2 genomes among re-infections and breakthrough COVID-19 cases revealed contiguous deletions (Δ85-90 and Δ167-174) that were rare among sequences deposited in GISAID at the time of this analysis. While they cannot yet be classified as new RDRs, the proximity of these regions to the antigenic supersite suggests that they may become more prevalent in the coming months and that deletions in these regions should be monitored for associations with future surges. The striking trend that the most frequently deleted NTD regions are proximal to a single antigenic supersite highlights the prominent role that host immunity has played in shaping the genomic evolution of SARS-CoV-2 from the beginning of this pandemic.

There are a few limitations of this study. First, the geographic distribution of sequences deposited in GISAID is not representative of the global population, with a majority of the sequences coming from the United States or the United Kingdom. Future genomic epidemiology studies would be improved by expanded sequencing efforts in other countries. Second, the identification of mutations associated with surges during early months of the pandemic is complicated by the relative paucity of whole genome sequencing data deposited during that time. Third, the GISAID data is not linked to any phenotypic information (e.g., disease severity) or relevant medical histories (e.g., comorbidities and vaccination status). Thus, while we are able to identify correlations between mutational prevalence and case surges, we cannot determine whether particular mutations are associated with more severe disease or are observed more frequently than expected by chance in vaccinated individuals. While the latter shortcoming is partially addressed by our independent whole genome sequencing of virus isolated from reinfected and vaccinated patients, this analysis was limited by the small size of the cohort (n=53) and the lack of corresponding antibody titer data.

Taken together, this study illustrates the value of intersecting the disparate fields of epidemiologic surveillance and genomic sequencing. With the COVID-19 vaccine rollout occurring at unprecedented rates, it is critical to rapidly identify emerging mutation patterns and then to characterize single mutations and combinations thereof for their impact on vaccine effectiveness. Looking forward, this dynamic process will require interdisciplinary collaboration among experts in genomics, clinical epidemiology, structural biology, and basic virology. We emphasize that to achieve these goals, we must expand sequencing efforts around the world and encourage the transparent linking of relevant phenotypic data to each deposited sequence.

Our study is extremely timely and has important therapeutic and public health policy implications. The repeated emergence deletions within an antigenic supersite should be considered when developing vaccines and biologics to counter the immuno-evasive strategies of SARS-CoV-2. From a public health standpoint, this study motivates the need to massively scale up whole-genome sequencing efforts globally and highlights the value of clinico-genomic studies which link sequence information to patient phenotypes, particularly in the setting of breakthrough infections.

### Materials and Methods

### Analysis of Publicly Deposited SARS-CoV-2 Genomic Sequences

9,299,506 SARS-CoV-2 genome sequences (with 1,601 unique lineages) were obtained from GISAID²⁶ (data retrieved from https://www.gisaid.org/ on 23 Mar. 2022) for the period of December 2019 to March 2022 across 213 geographical locations. The mutations were called using the Wuhan-Hu-1 sequence as reference (UniProt ID: P0DTC2). To filter out potential sequencing artifacts, we excluded mutations that were present in fewer than 100 sequences, resulting in 3378 unique Spike protein mutations.

### Identification of Surge-Associated SARS-CoV-2 Mutations

To identify mutations that have been temporally associated with surges in COVID-19 cases throughout the pandemic, we assessed monthly mutational prevalences and test positivity over three-month intervals in each country. For each of the 3378 mutations, the monthly mutational prevalence was computed for a given country as:

$$Mutational Prevalence = \frac{Number of sequences with a mutation in a given month}{Total number of sequences desposited in that month} \times 100$$

Positivity data for PCR tests was obtained from the OWID resource^{27,28} (retrieved from https://github.com/owid/covid-19-data/tree/master/public/data on Apr. 23, 2021). For each country, the monthly test positivity was calculated as:

$$Test Positivity = \frac{New cases in a given month ( smoothened )}{Total cases in that month ( smoothered )} \times 100$$

To identify surge-associated mutations, we classified the monthly mutational prevalence (for each mutation) and the monthly test positivity as increasing (monotonically), decreasing (monotonically), or mixed over sliding three-month intervals over the course of the pandemic. Any mutation which monotonically increased in prevalence over this interval in a country with a simultaneous monotonic increase in test positivity was defined as a “surge-associated mutation.” There were 116 such mutations.

### Comparison of Surge-Associated Mutations to Mutations in CDC Variants of Interest and Concern

In order to test the value of our method, we obtained the set of CDC variants of interest and concern as of Apr. 15, 2021⁸. At this time (April 2021), there were 5 variants of concern and 8 variants of interest, with no variants of high consequence. From the 13 classified variants, there 56 unique mutations listed, of which 25 were found only in variants of interest, 24 were found only in variants of concern, and 7 were found in both variants of interest and concern. After identifying the surge-associated mutations as described above, we determined the fraction of mutations comprising the CDC-classified variants which were captured by this approach.

### Assessment of Mutation Types for Enrichment of Surge-Associated Mutations

After identifying the 177 surge-associated mutations, we tested whether any of the contributing mutation types (deletions, insertions, or substitutions) were enriched for surge-associated mutations. To do so, we constructed a 3×2 table giving the number of surge-associated and non-surge-associated mutations in each category. To determine whether one or more groups showed a statistically significant enrichment, a chi-square p-value was calculated using the chisq.test function from the stats package (4.0.3) in R. Post-hoc tests were performed by considered constructing 2×2 contingency tables to compare each mutation type against all others. Then, odds ratios and their corresponding 95% confidence intervals were calculated using the fisher.test function from the stats package (version 4.0.3) in R.

### Identification of New Recurrent Deletion Regions in the Spike Protein

Recurrent deletion regions (RDRs) were previously defined as four sites within the NTD to which over 90% of all Spike protein deletions occurred, per the 146,795 SARS-CoV-2 sequences deposited in GISAID as of Oct. 24, 2020. To identify potential new RDRs that have emerged since this time, we first plotted the distribution of deletion counts for each amino acid (i.e. number of sequences in which deletion of the given amino acid was observed) in the Spike protein, considering all 9,299,506 sequences analyzed in this study. We calculated the 95th percentile of the deletion count distribution, which is 659. We then bucketed each residue R into categories (Yes, No, Possible) reflecting whether or not it should be considered as part of an RDR (i.e., a contiguous stretch of two or more amino acid residues which undergo deletion events more frequently than expected by chance) as follows (illustrated schematically in Table 2).

Once each residue was categorized in this way, then any residue P in the “Possible” category were subjected to further analysis to convert their labels into “Yes” or “No.” Specifically, we took a step-wise approach, walking in both directions from P until the first encounter of a residue categorized as “Yes” or “No” (i.e., other residues labeled as “Possible” were ignored). If a residue categorized as “Yes” was encountered before any residue categorized as “No” in either direction, then the “Possible” label was converted to “Yes.” If a residue categorized as “No” was encountered before any residue categorized as “Yes” in both directions, then the “Possible” label was converted to “Yes.”

With each residue categorized as “Yes” or “No”, we then simply merged the residue windows with consecutive “Yes” labels to define the updated set of Spike protein RDRs. We name the RDRs on the basis of the first and last amino acid residues contained within the region; for example, the RDR including residues C14, Q15, and V16 is defined as RDR₁₄₋₁₆.

### Temporal Analysis of Expansions in Recurrent Deletion Regions

To assess the expansion of regions undergoing deletions over time, we plotted a time series heatmap indicating the first time (month) at which a given deletion was identified across all GISAID sequences, and the number of sequences in which that deletion was detected in that month and all subsequent months. The residues plotted were defined based on the definition of RDRs provided above, which builds upon the regions defined previously¹⁵.

### Structural Analysis of SARS-CoV-2 Spike Protein

Structural analyses and illustrations were performed in PyMOL (version 2.3.4). The cryo-EM structure of the Spike protein characterizing the interaction with a neutralizing antibody 4A8 (PDB identifier: 7C2L), described by Chi et al.¹⁹, was retrieved from the PDB.

Whole Viral Genome Sequencing of SARS-CoV-2 Obtained from Individuals with Breakthrough Infections

This is a retrospective study of individuals who underwent polymerase chain reaction (PCR) testing for suspected SARS-CoV-2 infection at the Mayo Clinic and hospitals affiliated to the Mayo health system. This study was reviewed by the Mayo Clinic Institutional Review Board and determined to be exempt from human subjects research. Subjects were excluded if they did not have a research authorization on file.

SARS-CoV-2 RNA-positive upper respiratory tract swab specimens from patients with vaccine breakthrough or reinfection of COVID-19 were subjected to next-generation sequencing, using the commercially available Ion AmpliSeq SARS-CoV-2 Research Panel (Life Technologies Corp., South San Francisco, Calif.) based on the “sequencing by synthesis” method. The assay amplifies 237 sequences ranging from 125 to 275 base pairs in length, covering 99% of the SARS-CoV-2 genome. Viral RNA was first manually extracted and purified from these clinical specimens using MagMAX™ Viral/Pathogen Nucleic Acid Isolation Kit (Life Technologies Corp.), followed by automated reverse transcription-PCR (RT-PCR) of viral sequences, DNA library preparation (including enzymatic shearing, adapter ligation, purification, normalization), DNA template preparation, and sequencing on the automated Genexus™ Integrated Sequencer (Life Technologies Corp.) with the Genexus™ Software version 6.2.1. A no-template control and a positive SARS-CoV-2 control were included in each assay run for quality control purposes. Viral sequence data were assembled using the Iterative Refinement Meta-Assembler (IRMA) application (50% base substitution frequency threshold) to generate unamended plurality consensus sequences for analysis with the latest versions of the web-based application tools: Pangolin²⁹ for SARS-CoV-2 lineage assignment; Nextclade³⁰ for viral clade assignment, phylogenetic analysis, and S codon mutation calling, in comparison to the wild-type reference sequence of SARS-CoV-2 Wuhan-Hu-1 (lineage B, clade 19A).

### REFERENCES

1. COVID-19 map—johns Hopkins Coronavirus resource Center. https://coronavirus.jhu.edu/map.html.
2. Mallapaty, S. India's massive COVID surge puzzles scientists. Nature 592, 667-668 (2021).
3. Pawlowski, C. et al. FDA-authorized COVID-19 vaccines are effective per real-world evidence synthesized across a multi-state health system. MedRxiv (2021).
4. Corchado-Garcia, J. et al. Real-world effectiveness of Ad26.COV2.S adenoviral vector vaccine for COVID-19. doi:10.1101/2021.04.27.21256193.
5. Dagan, N. et al. BNT162b2 mRNA Covid-19 Vaccine in a Nationwide Mass Vaccination Setting. N. Engl. J. Med. 384, 1412-1423 (2021).
6. Hacisuleyman, E. et al. Vaccine Breakthrough Infections with SARS-CoV-2 Variants. N. Engl. J. Med. (2021) doi:10.1056/NEJMoa2105000.
7. Kustin, T. et al. Evidence for increased breakthrough rates of SARS-CoV-2 variants of concern in BNT162b2 mRNA vaccinated individuals. bioRxiv (2021) doi:10.1101/2021.04.06.21254882.
8. CDC. SARS-CoV-2 Variant Classifications and Definitions. https://www.cdc.gov/coronavirus/2019-ncov/cases-updates/variant-surveillance/variant-info.html (2021).
9. COVID-19 Virtual Press conference transcript—10 May 2021. https://www.who.int/publications/m/item/covid-19-virtual-press-conference-transcript---10-may-2021.
10. Barnes, C. O. et al. SARS-CoV-2 neutralizing antibody structures inform therapeutic strategies. Nature 588, 682-687 (2020).
11. Zost, S. J. et al. Rapid isolation and profiling of a diverse panel of human monoclonal antibodies targeting the SARS-CoV-2 spike protein. Nat. Med. 26, 1422-1427 (2020).
12. Liu, L. et al. Potent neutralizing antibodies against multiple epitopes on SARS-CoV-2 spike. Nature 584, 450-456 (2020).
13. McCallum, M. et al. N-terminal domain antigenic mapping reveals a site of vulnerability for SARS-CoV-2. Cell 184, 2332-2347.e16 (2021).
14. Cerutti, G. et al. Potent SARS-CoV-2 neutralizing antibodies directed against spike N-terminal domain target a single supersite. Cell Host Microbe 29, 819-833.e7 (2021).
15. McCarthy, K. R. et al. Recurrent deletions in the SARS-CoV-2 spike glycoprotein drive antibody escape. Science 371, 1139-1142 (2021).
16. PANGO lineages. https://cov-lineages.org/lineages/lineage_B.1.617.html.
17. Kuppalli, K. et al. India's COVID-19 crisis: a call for international action. Lancet (2021) doi:10.1016/50140-6736(21)01121-1.
18. Taylor, L. Covid-19: Spike in cases in Chile is blamed on people mixing after first vaccine shot. BMJ 373, n1023 (2021).
19. Chi, X. et al. A neutralizing human antibody binds to the N-terminal domain of the Spike protein of SARS-CoV-2. Science 369, 650-655 (2020).
20. Anand, P., Puranik, A., Aravamudan, M., Venkatakrishnan, A. J. & Soundararajan, V. SARS-CoV-2 strategically mimics proteolytic activation of human ENaC. Elife 9, (2020).
21. Johnson, B. A. et al. Loss of furin cleavage site attenuates SARS-CoV-2 pathogenesis. Nature 591, 293-299 (2021).
22. Coutard, B. et al. The spike glycoprotein of the new coronavirus 2019-nCoV contains a furin-like cleavage site absent in CoV of the same clade. Antiviral Res. 176, 104742 (2020).
23. Walls, A. C. et al. Structure, Function, and Antigenicity of the SARS-CoV-2 Spike Glycoprotein. Cell 181, 281-292.e6 (2020).
24. Liu, Y. et al. Neutralizing Activity of BNT162b2-Elicited Serum. N. Engl. J. Med. 384, 1466-1468 (2021).
25. Wang, P. et al. Antibody resistance of SARS-CoV-2 variants B.1.351 and B.1.1.7. Nature 593, 130-135 (2021).
26. Shu, Y. & McCauley, J. GISAID: Global initiative on sharing all influenza data—from vision to reality. Euro Surveill. 22, (2017).
27. Our World in Data. https://ourworldindata.org/.
28. Hasell, J. et al. A cross-country database of COVID-19 testing. Sci Data 7, 345 (2020).
29. COG-UK. https://pangolin.cog-uk.io/.
30. Nextclade.https://clades.nextstrain.org/.

### Table(s)

### Table 1:

Mutations correlated with recent increased test positivity rate over the three-month period starting between February 2021 and April, 2021. We ensured that these mutations are prevalent in at least 5% of the number of sequences deposited within this time period in GISAID. A minimum cut-off of 5% test positivity within this three-month window was also applied to ensure we capture surges with relevant magnitude associated with it. Only the top five mutations that were observed to have maximum change in their prevalence % (min−max) over the three-month period. The test positivity rate observed across these three-months in different countries are also shown.

| | No. of | | | |
|---|---|---|---|---|
| | mtns, | | | Test positivity |
| | correlated | | Mutations with high magnitude | rate change |
| | with | | changes Between February 2021 | Between February |
| Country | surge | Mutations | to April 2021 | 2021 to April 2021 |
| Poland | 6 | P681H, N501Y, A570D, | A570D: (58.135, 88.857, 98.733) | (13.982, 25.464, |
| | | T716I, D1118H, S982A | N501Y: (58.392, 91.302, 98.804) | 26.792) |
| | | | S982A: (58.264, 91.045, 98.663) | |
| | | | D1118H: (58.135, 91.148, 98.452) | |
| | | | T716I: (58.585, 91.173, 98.663) | |
| Bangladesh | 11 | L18F, E484K, A701V, | A701V : (15.094, 66.667, 82.569) | (3.056, 6.283, |
| | | T95I, K417N, D80A, | D80A: (9.434, 64.368, 72.477) | 20.161) |
| | | ΔA243, D215G, ΔL244, | D215G: (11.321, 60.92, 71.56) | |
| | | ΔL242, D796H | K417N: (13.208, 65.517, 73.394) | |
| | | | ΔA243: (13.208, 62.069, 70.642) | |
| Belgium | 21 | D614G, P681H, N501Y, | ΔH69: (44.252, 70.93, 82.939) | (4.762, 6.051, |
| | | ΔV70, ΔH69, A570D, T716I, | ΔV70: (44.355, 70.912, 82.939) | 8.434) |
| | | ΔY144, D1118H, S982A, | ΔY144: (43.967, 70.764, 82.164) | |
| | | L18F, E484K, L5F, | N501Y: (59.425, 87.339, 93.912) | |
| | | D138Y, V1176F, P26S, H655Y, | D1118H: (49.379, 75.581, 82.745) | |
| | | T1027I, T20N, K417T, R190S | | |
| Chile | 36 | P681H, N501YΔ, V70, ΔH69, | N501Y: (8.547, 32.45, 44.493) | (7.265, 8.632, |
| | | A570d, T716I, ΔY144, | T1027I: (2.564, 24.503, 36.123) | 11.218) |
| | | D1118H, S982A, L18F, | P26S: (2.564, 23.51, 35.242) | |
| | | E484K, D138Y, V1176F, | R190S: (2.564, 23.179, 35.242) | |
| | | P26S, H655Y, T1027I, T20N, | T20N: (2.564, 21.192, 34.802) | |
| | | K417T, R190S, T859N, | | |
| | | G75V, T76I, F490S, ΔS71, | | |
| | | L452Q, ΔR246, D253N, | | |
| | | ΔS247, ΔY248, ΔG252, | | |
| | | ΔP251, ΔL249, ΔT250, | | |
| | | ΔG72, ΔI68, ΔT73 | | |
| France | 18 | D614G, P681H, N501Y, ΔV70, | ΔY144: (50.812, 70.954, 80.552) | (6.235, 7.116, |
| | | ΔH69, A570D, T716I, | S982A: (51.879, 72.911, 80.736) | 9.555) |
| | | ΔY144, D1118H, S982A, | T716I: (52.606, 74.113, 81.172) | |
| | | E484K, A701V, S98F, D80A, | A570D: (53.067, 74.956, 81.31) | |
| | | ΔA243, D215G, ΔL244, ΔL242 | D1118H: (51.685, 72.737, 79.885) | |
| India | 13 | D14G, A222V, L452R, | P681R: (26.236, 43.642, 68.937) | (1.76, 2.896, |
| | | T478K, P681R, G142D, Q1071H, | L452R: (26.954, 46.773, 68.392) | 11.276) |
| | | D950N, T19R, ΔR158, ΔF157, | T19R: (1.116, 8.498, 30.79) | |
| | | E156G, H1101D | T478K: (0.239, 7.668, 29.155) | |
| | | | D950N : (1.116, 7.476, 25.613) | |
| Sweden | 11 | D614G, P681H, N501Y, | T716I: (58.007, 88.597, 96.863) | (10.118, 10.486, |
| | | ΔV70, ΔH69, A570D, T716I, | A570D: (57.952, 88.614, 96.787) | 14.479) |
| | | ΔY144, D1118H, S982A, T20I | D1118H: (57.889, 88.58, 96.021) | |
| | | | S982A: (57.897, 88.555, 96.021) | |
| | | | P681H: (59.337, 88.368, 96.94) | |

### Table 2:

Schematic representation of the decision schema for considering a residue R to be a part of a RDR. Deletion count of <=687 is represented by X. Deletion count of >=688 is represented by ✓.

| | | | | | Should Residue R |
|---|---|---|---|---|---|
| Deletion count | | | | | be considered part |
| R - 2 | R - 1 | Residue R | R + 1 | R + 2 | of a RDR? |
| | X | X | | | No |
| | | X | X | | No |
| | ✓ | ✓ | | | Yes |
| | | ✓ | ✓ | | Yes |
| | ✓ | | ✓ | ✓ | Yes |
| ✓ | ✓ | | ✓ | | Yes |
| X | X | ✓ | | ✓ | Possible |
| ✓ | | ✓ | X | X | Possible |
| | ✓ | X | ✓ | | Possible |

### Table 3:

List of GISAID accession IDs with the same recurrent deletions observed as seen in the vaccine breakthrough patients.

| Accession ID | Collection date | Location |
|---|---|---|
| EPI_ISL_3543445 | 12 Aug. 2020 | North America/USA/Idaho |
| EPI_ISL_3923182 | 14 Aug. 2020 | North America/USA/Idaho/Ada |
| EPI_ISL_6938332 | 15 Oct. 2020 | North America/USA/Montana/Flathead County |
| EPI_ISL_1493068 | 25 Jan. 2021 | North America/USA/Oregon/Washington County |
| EPI_ISL_3032084 | 27 Jan. 2021 | North America/USA/Rhode Island |
| EPI_ISL_1236478 | 26 Feb. 2021 | North America/USA/Texas/Houston |
| EPI_ISL_5388899 | 31 Mar. 2021 | North America/USA/Oregon/Benton County |
| EPI_ISL_4572606 | 13 Apr. 2021 | North America/USA/Mississippi |
| EPI_ISL_2039575 | 16 Apr. 2021 | North America/USA/New Jersey |
| EPI_ISL_1840693 | 17 Apr. 2021 | North America/USA/Texas |
| EPI_ISL_2254264 | 18 Apr. 2021 | North America/USA/Michigan |
| EPI_ISL_2377899 | 29 Apr. 2021 | North America/USA/Ohio |
| EPI_ISL_2023473 | 8 May 2021 | North America/USA/Oregon |
| EPI_ISL_2550761 | 18 May 2021 | North America/USA/Maryland/MDH |
| EPI_ISL_3243026 | 18 May 2021 | North America/USA/New York |
| EPI_ISL_2557914 | 26 May 2021 | North America/USA/Oregon/Jackson County |
| EPI_ISL_2689086 | 11 Jun. 2021 | North America/USA/Maryland |
| EPI_ISL_2955582 | 28 Jun. 2021 | North America/USA/Maryland |
| EPI_ISL_2933949 | 1 Jul. 2021 | North America/USA/Oregon/Lane County |
| EPI_ISL_2955406 | 6 Jul. 2021 | North America/USA/Oregon/Deschutes County |
| EPI_ISL_3161298 | 6 Jul. 2021 | North America/USA/Louisiana |
| EPI_ISL_3134170 | 13 Jul. 2021 | North America/USA/Texas |
| EPI_ISL_3241903 | 15 Jul. 2021 | North America/USA/Maryland |
| EPI_ISL_3241913 | 16 Jul. 2021 | North America/USA/Maryland |
| EPI_ISL_9846980 | 18 Jul. 2021 | North America/USA/California/San Mateo County |
| EPI_ISL_3548380 | 25 Jul. 2021 | North America/USA/Maryland |
| EPI_ISL_4572632 | 30 Jul. 2021 | North America/USA/Mississippi |
| EPI_ISL_3588762 | 1 Aug. 2021 | North America/USA/Idaho/Ada |
| EPI_ISL_3743152 | 2 Aug. 2021 | North America/USA/Idaho/Ada |
| EPI_ISL_4572661 | 3 Aug. 2021 | North America/USA/Mississippi |
| EPI_ISL_4467836 | 6 Aug. 2021 | North America/USA/Utah |
| EPI_ISL_3923035 | 12 Aug. 2021 | North America/USA/Idaho/Ada |
| EPI_ISL_4211354 | 13 Aug. 2021 | North America/USA/Maryland |
| EPI_ISL_3922902 | 15 Aug. 2021 | North America/USA/Idaho/Ada |
| EPI_ISL_4211360 | 15 Aug. 2021 | North America/USA/Maryland |
| EPI_ISL_4624971 | 20 Aug. 2021 | North America/USA/Maryland/MDH |
| EPI_ISL_4659943 | 21 Aug. 2021 | North America/USA/Maryland |
| EPI_ISL_4211346 | 23 Aug. 2021 | North America/USA/Maryland |
| EPI_ISL_3824617 | 25 Aug. 2021 | North America/USA/Georgia |
| EPI_ISL_3824616 | 25 Aug. 2021 | North America/USA/Georgia |
| EPI_ISL_3824614 | 25 Aug. 2021 | North America/USA/Georgia |
| EPI_ISL_4929936 | 31 Aug. 2021 | North America/USA/Maryland |
| EPI_ISL_5233415 | 2 Sep. 2021 | North America/USA/Minnesota |
| EPI_ISL_4659931 | 13 Sep. 2021 | North America/USA/Maryland |
| EPI_ISL_5511690 | 21 Sep. 2021 | North America/USA/Idaho |
| EPI_ISL_6067828 | 6 Oct. 2021 | North America/USA/Wisconsin |
| EPI_ISL_7547075 | 11 Oct. 2021 | North America/USA/Oregon/Linn County |
| EPI_ISL_10842713 | 3 Nov. 2021 | North America/USA/Ohio |
| EPI_ISL_10842290 | 22 Nov. 2021 | North America/USA/Ohio |
| EPI_ISL_7855059 | 9 Dec. 2021 | North America/USA/Louisiana |
| EPI_ISL_7855060 | 9 Dec. 2021 | North America/USA/Louisiana |
| EPI_ISL_7856051 | 13 Dec. 2021 | North America/USA/Louisiana |
| EPI_ISL_11170655 | 14 Dec. 2021 | North America/USA/California |
| EPI_ISL_8083592 | 15 Dec. 2021 | North America/USA/Louisiana/Calcasieu Parish |
| EPI_ISL_8083594 | 15 Dec. 2021 | North America/USA/Louisiana/Red River Parish |
| EPI_ISL_8083595 | 15 Dec. 2021 | North America/USA/Louisiana/Jefferson Parish |
| EPI_ISL_8162941 | 22 Dec. 2021 | North America/USA/Kansas/Spring Hill |
| EPI_ISL_9203222 | 4 Jan. 2022 | North America/USA/Idaho |
| EPI_ISL_9095808 | 5 Jan. 2022 | North America/USA/Idaho |
| EPI_ISL_10570882 | 6 Jan. 2022 | North America/USA/Colorado |
| EPI_ISL_9095825 | 7 Jan. 2022 | North America/USA/Idaho |
| EPI_ISL_9352268 | 10 Jan. 2022 | North America/USA/Idaho |
| EPI_ISL_8751566 | 10 Jan. 2022 | North America/USA/New York |
| EPI_ISL_9095855 | 10 Jan. 2022 | North America/USA/Idaho |
| EPI_ISL_9637035 | 11 Jan. 2022 | North America/USA/Idaho |
| EPI_ISL_9203214 | 12 Jan. 2022 | North America/USA/Idaho |
| EPI_ISL_9203199 | 13 Jan. 2022 | North America/USA/Idaho |
| EPI_ISL_9203176 | 13 Jan. 2022 | North America/USA/Idaho |
| EPI_ISL_9348957 | 18 Jan. 2022 | North America/USA/Idaho |
| EPI_ISL_9348953 | 18 Jan. 2022 | North America/USA/Idaho |
| EPI_ISL_9348952 | 18 Jan. 2022 | North America/USA/Idaho |
| EPI_ISL_9410506 | 18 Jan. 2022 | North America/USA/Idaho/Ada County |
| EPI_ISL_9153000 | 18 Jan. 2022 | North America/USA/Idaho |
| EPI_ISL_9152991 | 18 Jan. 2022 | North America/USA/Idaho |
| EPI_ISL_9152995 | 18 Jan. 2022 | North America/USA/Idaho |
| EPI_ISL_9152982 | 18 Jan. 2022 | North America/USA/Idaho |
| EPI_ISL_9149640 | 18 Jan. 2022 | North America/USA/Louisiana |
| EPI_ISL_9143287 | 18 Jan. 2022 | North America/USA/Louisiana/Jefferson Parish |
| EPI_ISL_9520550 | 19 Jan. 2022 | North America/USA/Idaho |
| EPI_ISL_9142380 | 19 Jan. 2022 | North America/USA/Louisiana |
| EPI_ISL_9410534 | 19 Jan. 2022 | North America/USA/Idaho/Ada County |
| EPI_ISL_9410545 | 19 Jan. 2022 | North America/USA/Idaho/Ada County |
| EPI_ISL_9487071 | 19 Jan. 2022 | North America/USA/Idaho |
| EPI_ISL_9348970 | 19 Jan. 2022 | North America/USA/Idaho |
| EPI_ISL_9348938 | 19 Jan. 2022 | North America/USA/Idaho |
| EPI_ISL_9348991 | 19 Jan. 2022 | North America/USA/Idaho |
| EPI_ISL_9437788 | 19 Jan. 2022 | North America/USA/Idaho |
| EPI_ISL_9520548 | 20 Jan. 2022 | North America/USA/Idaho |
| EPI_ISL_10057504 | 20 Jan. 2022 | North America/USA/Idaho |
| EPI_ISL_9573361 | 24 Jan. 2022 | North America/USA/Louisiana |
| EPI_ISL_9571843 | 24 Jan. 2022 | North America/USA/Louisiana/Orleans Parish |
| EPI_ISL_9571837 | 24 Jan. 2022 | North America/USA/Louisiana/OrleansParish |
| EPI_ISL_9570709 | 24 Jan. 2022 | North America/USA/Louisiana/Orleans Parish |
| EPI_ISL_9570719 | 24 Jan. 2022 | North America/USA/Louisiana |
| EPI_ISL_10057489 | 25 Jan. 2022 | North America/USA/Idaho |
| EPI_ISL_9551901 | 25 Jan. 2022 | North America/USA/Idaho |
| EPI_ISL_9551927 | 25 Jan. 2022 | North America/USA/Idaho |
| EPI_ISL_9551930 | 25 Jan. 2022 | North America/USA/Idaho |
| EPI_ISL_10057508 | 25 Jan. 2022 | North America/USA/Idaho |
| EPI_ISL_10057496 | 25 Jan. 2022 | North America/USA/Idaho |
| EPI_ISL_10057497 | 25 Jan. 2022 | North America/USA/Idaho |
| EPI_ISL_9873151 | 26 Jan. 2022 | North America/USA/Idaho |
| EPI_ISL_10082644 | 27 Jan. 2022 | North America/USA/Idaho/Canyon |
| EPI_ISL_9942059 | 6 Feb. 2022 | North America/USA/New York |
| EPI_ISL_10068075 | 7 Feb. 2022 | North America/USA/Louisiana |
| EPI_ISL_10068325 | 7 Feb. 2022 | North America/USA/Louisiana |
| EPI_ISL_10063454 | 7 Feb. 2022 | North America/USA/Louisiana |
| EPI_ISL_10064537 | 7 Feb. 2022 | North America/USA/Louisiana |
| EPI_ISL_10067496 | 7 Feb. 2022 | North America/USA/Louisiana |
| EPI_ISL_10556645 | 16 Feb. 2022 | North America/USA/Louisiana |
| EPI_ISL_10621433 | 17 Feb. 2022 | North America/USA/Louisiana |
| EPI_ISL_10556725 | 17 Feb. 2022 | North America/USA/Louisiana |
| EPI_ISL_10402769 | 17 Feb. 2022 | North America/USA/Idaho |
| EPI_ISL_10556844 | 17 Feb. 2022 | North America/USA/Louisiana |
| EPI_ISL_10556621 | 17 Feb. 2022 | North America/USA/Louisiana |
| EPI_ISL_10619979 | 17 Feb. 2022 | North America/USA/Louisiana |
| EPI_ISL_10623276 | 17 Feb. 2022 | North America/USA/Louisiana |
| EPI_ISL_10396826 | 18 Feb. 2022 | North America/USA/New York |
| EPI_ISL_10623306 | 18 Feb. 2022 | North America/USA/Louisiana |
| EPI_ISL_11171641 | 22 Feb. 2022 | North America/USA/New York |
| EPI_ISL_11171672 | 23 Feb. 2022 | North America/USA/New York |
| EPI_ISL_11191720 | 28 Feb. 2022 | North America/USA/Louisiana/Rapides |
| EPI_ISL_11191724 | 28 Feb. 2022 | North America/USA/Louisiana/Rapides |
| EPI_ISL_11191719 | 28 Feb. 2022 | North America/USA/Louisiana/East Baton Rouge |
| EPI_ISL_11096053 | 1 Mar. 2022 | North America/USA/Idaho |
| EPI_ISL_11172899 | 2 Mar. 2022 | North America/USA/New York |
| EPI_ISL_11191732 | 3 Mar. 2022 | North America/USA/Louisiana |
| EPI_ISL_11191729 | 3 Mar. 2022 | North America/USA/Louisiana |

### Table 4:

All the mutations in the spike protein that have positive correlation with the test positivity percentage across the complete timeline of pandemic in India has been tabulated here. Following are the expansion of the abbreviations used in the table header—Total Seqs. Dep.: Total number of sequences deposited in the particular month in India. Test Pos. %: Test positivity percentage, Mut Prev. %: Mutation prevalence percentage, Rho (Pearson) Mut Prev. % vs Test Pos. %: The Pearson correlation Rho value between test positivity and mutational prevalence, Test pos. List: test positivity percentage over the window of 3 months, Mut Prev. List: mutation prevalence percentage over the window of 3 months, MaxΔ Mut Prev.: maximum difference in the mutational prevalence percentage observed over the window of 3 months.

| | | Total | Test | Mutation | Rho (Pearson) | Test Pos. | Mut Prev. | Max Δ Mut |
|---|---|---|---|---|---|---|---|---|
| Spike | | Seqs. | Positivity | Prevalence | Mut Prev. % vs. | List (3 | List (3 | Prev. |
| Mutation | Month | Dep. | % | % | Test Pos. % | months) | months) | (3 months) |
| D614G | June 2020 | 1092 | 7.694 | 98.81 | 0.746 | (4.516, | (85.859, | 12.951 |
| | | | | | | 4.919, | 96.076, | |
| | | | | | | 7.694) | 98.81) | |
| D614G | April 2021 | 367 | 11.276 | 100 | 0.823 | (1.76, | (99.043, | 0.957 |
| | | | | | | 2.896, | 99.681, | |
| | | | | | | 11.276) | 100.0) | |
| A222V | April 2021 | 367 | 11.276 | 8.174 | 1 | (1.76, | (0.319, | 7.855 |
| | | | | | | 2.896, | 1.214, | |
| | | | | | | 11.276) | 8.174) | |
| L452R | April 2021 | 367 | 11.276 | 68.392 | 0.925 | (1.76, | (26.954, | 41.438 |
| | | | | | | 2.896, | 46.773, | |
| | | | | | | 11.276) | 68.392) | |
| T478K | April 2021 | 367 | 11.276 | 29.155 | 0.99 | (1.76, | (0.239, | 28.916 |
| | | | | | | 2.896, | 7.668, | |
| | | | | | | 11.276) | 29.155) | |
| P681R | April 2021 | 367 | 11.276 | 68.937 | 0.953 | (1.76, | (26.236, | 42.701 |
| | | | | | | 2.896, | 43.642, | |
| | | | | | | 11.276) | 68.937) | |
| L54F | June 20 | 1092 | 7.694 | 8.7 | 0.999 | (4.516, | (0.337, | 8.363 |
| | | | | | | 4.919, | 1.107, 8.7) | |
| | | | | | | 7.694) | | |
| G142D | April 2021 | 367 | 11.276 | 23.433 | 0.615 | (1.76, | (12.281, | 11.152 |
| | | | | | | 2.896, | 23.067, | |
| | | | | | | 11.276) | 23.433) | |
| Q1071H | April 2021 | 367 | 11.276 | 31.608 | 0.72 | (1.76, | (17.225, | 14.383 |
| | | | | | | 2.896, | 29.01, | |
| | | | | | | 11.276) | 31.608) | |
| D950N | April 2021 | 367 | 11.276 | 25.613 | 0.99 | (1.76, | (1.116, | 24.497 |
| | | | | | | 2.896, | 7.476, | |
| | | | | | | 11.276) | 25.613) | |
| T19R | April 2021 | 367 | 11.276 | 30.79 | 0.991 | (1.76, | (1.116, | 29.674 |
| | | | | | | 2.896, | 8.498, | |
| | | | | | | 11.276) | 30.79) | |
| ΔR158 | April 2021 | 367 | 11.276 | 14.986 | 0.997 | (1.76, | (1.116, | 13.87 |
| | | | | | | 2.896, | 1.534, | |
| | | | | | | 11.276) | 14.986) | |
| ΔF157I | April 2021 | 367 | 11.276 | 14.986 | 0.997 | (1.76, | (1.116, | 13.87 |
| | | | | | | 2.896, | 1.534, | |
| | | | | | | 11.276) | 14.986) | |
| E156G | April 2021 | 367 | 11.276 | 14.986 | 0.997 | (1.76, | (1.116, | 13.87 |
| | | | | | | 2.896, | 1.534, | |
| | | | | | | 11.276) | 14.986) | |
| H1101D | April 2021 | 367 | 11.276 | 14.441 | 0.845 | (1.76, | (6.459, | 7.982 |
| | | | | | | 2.896, | 11.502, | |
| | | | | | | 11.276) | 14.441) | |

### Table 5:

All the mutations in the spike protein that have positive correlation with the test positivity percentage across the complete timeline of pandemic in Chile has been tabulated here. Following are the expansion of the abbreviations used in the table header—Total Seqs. Dep.: Total number of sequences deposited in the particular month in Chile. Test Pos. %: Test positivity percentage, Mut Prev. %: Mutation prevalence percentage, Rho (Pearson) Mut Prev. % vs Test Pos. %: The Pearson correlation Rho value between test positivity and mutational prevalence, Test pos. List: test positivity percentage over the window of 3 months, Mut Prev. List: mutation prevalence percentage over the window of 3 months, MaxΔ Mut Prev.: maximum difference in the mutational prevalence percentage observed over the window of 3 months.

| | | | | | Rho | | | |
|---|---|---|---|---|---|---|---|---|
| | | | | | (Pearson) | | | |
| | | | | | Mut | | | |
| | | Total | Test | Mut | Prev. % vs. | Test Pos. | Mut Prev. | Max Δ Mut |
| Spike | | Seqs. | Positivity | Prevalence | Test | List (3 | List (3 | Prev. |
| Mutation | Month | Deposited | % | % | Pos. % | months) | months) | (3 months) |
| P681H | March 2021 | 302 | 8.632 | 9.603 | 0.581 | (7.19, | (6.034, | 1.442 |
| | | | | | | 7.265, | 9.402, | |
| | | | | | | 8.632) | 9.603) | |
| P681H | April 2021 | 227 | 11.218 | 9.692 | 0.923 | (7.265, | (9.402, | 3.953 |
| | | | | | | 8.632, | 9.603, | |
| | | | | | | 11.218) | 9.692) | |
| N501Y | March 2021 | 302 | 8.632 | 32.45 | 0.998 | (7.19, | (5.172, | 1.442 |
| | | | | | | 7.265, | 8.547, | |
| | | | | | | 8.632) | 32.45) | |
| N501Y | April 2021 | 227 | 11.218 | 44.493 | 0.934 | (7.265, | (8.547, | 3.953 |
| | | | | | | 8.632, | 32.45, | |
| | | | | | | 11.218) | 44.493) | |
| ΔV70 | March 2021 | 302 | 8.632 | 10.927 | 0.942 | (7.19, | (4.31, | 1.442 |
| | | | | | | 7.265, | 6.838, | |
| | | | | | | 8.632) | 10.927) | |
| ΔV70 | April 2021 | 227 | 11.218 | 14.537 | 0.978 | (7.265, | (6.838, | 3.953 |
| | | | | | | 8.632, | 10.927, | |
| | | | | | | 11.218) | 14.537) | |
| ΔH69 | March 2021 | 302 | 8.632 | 10.927 | 0.942 | (7.19, | (4.31, | 1.442 |
| | | | | | | 7.265, | 6.838, | |
| | | | | | | 8.632) | 10.927) | |
| ΔH69 | April 2021 | 227 | 11.218 | 14.537 | 0.978 | (7.265, | (6.838, | 3.953 |
| | | | | | | 8.632, | 10.927, | |
| | | | | | | 11.218) | 14.537) | |
| A570D | March 2021 | 302 | 8.632 | 7.616 | 0.824 | (7.19, | (3.448, | 1.442 |
| | | | | | | 7.265, | 5.983, | |
| | | | | | | 8.632) | 7.616) | |
| A570D | April 2021 | 227 | 11.218 | 9.251 | 0.985 | (7.265, | (5.983, | 3.953 |
| | | | | | | 8.632, | 7.616, | |
| | | | | | | 11.218) | 9.251) | |
| T716I | March 2021 | 302 | 8.632 | 8.94 | 0.863 | (7.19, | (4.31, | 1.442 |
| | | | | | | 7.265, | 6.838, | |
| | | | | | | 8.632) | 8.94) | |
| T716I | April 2021 | 227 | 11.218 | 9.251 | 0.836 | (7.265, | (6.838, | 3.953 |
| | | | | | | 8.632, | 8.94, | |
| | | | | | | 11.218) | 9.251) | |
| ΔΥ144 | March 2021 | 302 | 8.632 | 7.947 | 0.591 | (7.19, | (4.31, | 1.442 |
| | | | | | | 7.265, | 7.692, | |
| | | | | | | 8.632) | 7.947) | |
| ΔΥ144 | April 2021 | 227 | 11.218 | 9.251 | 0.981 | (7.265, | (7.692, | 3.953 |
| | | | | | | 8.632, | 7.947, | |
| | | | | | | 11.218) | 9.251) | |
| D1118H | March 2021 | 302 | 8.632 | 7.947 | 0.852 | (7.19, | (3.448, | 1.442 |
| | | | | | | 7.265, | 5.983, | |
| | | | | | | 8.632) | 7.947) | |
| D1118H | April 2021 | 227 | 11.218 | 9.251 | 0.958 | (7.265, | (5.983, | 3.953 |
| | | | | | | 8.632, | 7.947, | |
| | | | | | | 11.218) | 9.251) | |
| S982A | March 2021 | 302 | 8.632 | 7.947 | 0.852 | (7.19, | (3.448, | 1.442 |
| | | | | | | 7.265, | 5.983, | |
| | | | | | | 8.632) | 7.947) | |
| S982A | April 2021 | 227 | 11.218 | 9.251 | 0.958 | (7.265, | (5.983, | 3.953 |
| | | | | | | 8.632, | 7.947, | |
| | | | | | | 11.218) | 9.251) | |
| L18F | April 2021 | 227 | 11.218 | 35.683 | 0.954 | (7.265, | (4.274, | 3.953 |
| | | | | | | 8.632, | 23.51, | |
| | | | | | | 11.218) | 35.683) | |
| L452R | February 2021 | 117 | 7.265 | 3.419 | 0.759 | (4.518, | (0.943, | 2.747 |
| | | | | | | 7.19, | 1.724, | |
| | | | | | | 7.265) | 3.419) | |
| E484K | April 2021 | 227 | 11.218 | 36.123 | 0.933 | (7.265, | (5.983, | 3.953 |
| | | | | | | 8.632, | 26.159, | |
| | | | | | | 11.218) | 36.123) | |
| L5F | February 2021 | 117 | 7.265 | 3.419 | 0.759 | (4.518, | (0.943, | 2.747 |
| | | | | | | 7.19, | 1.724, | |
| | | | | | | 7.265) | 3.419) | |
| D138Y | February 2021 | 117 | 7.265 | 2.564 | 0.875 | (5.103, | (0.917, | 2.162 |
| | | | | | | 7.19, | 1.724, | |
| | | | | | | 7.265) | 2.564) | |
| D138Y | March 2021 | 302 | 8.632 | 23.179 | 1 | (7.19, | (1 -724, | 1.442 |
| | | | | | | 7.265, | 2.564, | |
| | | | | | | 8.632) | 23.179) | |
| D138Y | April 2021 | 227 | 11.218 | 34.361 | 0.941 | (7.265, | (2.564, | 3.953 |
| | | | | | | 8.632, | 23.179, | |
| | | | | | | 11.218) | 34.361) | |
| V1176F | April 2021 | 227 | 11.218 | 35.242 | 0.924 | (7.265, | (5.983, | 3.953 |
| | | | | | | 8.632, | 26.159, | |
| | | | | | | 11.218) | 35.242) | |
| P26S | March 2021 | 302 | 8.632 | 23.51 | 1 | (7.19, | (1 -724, | 1.442 |
| | | | | | | 7.265, | 2.564, | |
| | | | | | | 8.632) | 23.51) | |
| P26S | April 2021 | 227 | 11.218 | 35.242 | 0.944 | (7.265, | (2.564, | 3.953 |
| | | | | | | 8.632, | 23.51, | |
| | | | | | | 11.218) | 35.242) | |
| H655Y | March 2021 | 302 | 8.632 | 26.159 | 0.996 | (7.19, | (2.586, | 1.442 |
| | | | | | | 7.265, | 5.983, | |
| | | | | | | 8.632) | 26.159) | |
| H655Y | April 2021 | 227 | 11.218 | 34.802 | 0.92 | (7.265, | (5.983, | 3.953 |
| | | | | | | 8.632, | 26.159, | |
| | | | | | | 11.218) | 34.802) | |
| T1027I | March 2021 | 302 | 8.632 | 24.503 | 1 | (7.19, | (1 -724, | 1.442 |
| | | | | | | 7.265, | 2.564, | |
| | | | | | | 8.632) | 24.503) | |
| T1027I | April 2021 | 227 | 11.218 | 36.123 | 0.939 | (7.265, | (2.564, | 3.953 |
| | | | | | | 8.632, | 24.503, | |
| | | | | | | 11.218) | 36.123) | |
| T20N | March 2021 | 302 | 8.632 | 21.192 | 1 | (7.19, | (0.862, | 1.442 |
| | | | | | | 7.265, | 2.564, | |
| | | | | | | 8.632) | 21.192) | |
| T20N | April 2021 | 227 | 11.218 | 34.802 | 0.965 | (7.265, | (2.564, | 3.953 |
| | | | | | | 8.632, | 21.192, | |
| | | | | | | 11.218) | 34.802) | |
| K417T | April 2021 | 227 | 11.218 | 22.467 | 1 | (7.19, | (1.724, | 4.028 |
| | | | | | | 8.632, | 9.603, | |
| | | | | | | 11.218) | 22.467) | |
| R190S | March 2021 | 302 | 8.632 | 23.179 | 1 | (7.19, | (0.862, | 1.442 |
| | | | | | | 7.265, | 2.564, | |
| | | | | | | 8.632) | 23.179) | |
| R190S | April 2021 | 227 | 11.218 | 35.242 | 0.947 | (7.265, | (2.564, | 3.953 |
| | | | | | | 8.632, | 23.179, | |
| | | | | | | 11.218) | 35.242) | |
| T859N | March 2021 | 302 | 8.632 | 31.126 | 0.99 | (7.19, | (0.862, | 1.442 |
| | | | | | | 7.265, | 6.838, | |
| | | | | | | 8.632) | 31.126) | |
| T859N | April 2021 | 227 | 11.218 | 34.361 | 0.83 | (7.265, | (6.838, | 3.953 |
| | | | | | | 8.632, | 31.126, | |
| | | | | | | 11.218) | 34.361) | |
| G75V | March 2021 | 302 | 8.632 | 27.152 | 0.985 | (7.19, | (0.862, | 1.442 |
| | | | | | | 7.265, | 6.838, | |
| | | | | | | 8.632) | 27.152) | |
| G75V | April 2021 | 227 | 11.218 | 28.634 | 0.803 | (7.265, | (6.838, | 3.953 |
| | | | | | | 8.632, | 27.152, | |
| | | | | | | 11.218) | 28.634) | |
| T76I | March 2021 | 302 | 8.632 | 27.483 | 0.991 | (7.19, | (0.862, | 1.442 |
| | | | | | | 7.265, | 5.983, | |
| | | | | | | 8.632) | 27.483) | |
| T76I | April 2021 | 227 | 11.218 | 29.075 | 0.803 | (7.265, | (5.983, | 3.953 |
| | | | | | | 8.632, | 27.483, | |
| | | | | | | 11.218) | 29.075) | |
| F490S | March 2021 | 302 | 8.632 | 31.126 | 0.99 | (7.19, | (0.862, | 1.442 |
| | | | | | | 7.265, | 6.838, | |
| | | | | | | 8.632) | 31.126) | |
| F490S | April 2021 | 227 | 11.218 | 34.361 | 0.83 | (7.265, | (6.838, | 3.953 |
| | | | | | | 8.632, | 31.126, | |
| | | | | | | 11.218) | 34.361) | |
| ΔS71 | April 2021 | 227 | 11.218 | 5.286 | 0.948 | (7.265, | (0.855, | 3.953 |
| | | | | | | 8.632, | 3.642, | |
| | | | | | | 11.218) | 5.286) | |
| L452Q | March 2021 | 302 | 8.632 | 31.788 | 0.986 | (7.19, | (0.862, | 1.442 |
| | | | | | | 7.265, | 7.692, | |
| | | | | | | 8.632) | 31.788) | |
| L452Q | April 2021 | 227 | 11.218 | 34.361 | 0.818 | (7.265, | (7.692, | 3.953 |
| | | | | | | 8.632, | 31.788, | |
| | | | | | | 11.218) | 34.361) | |
| G1167A | January 2021 | 116 | 7.19 | 35.345 | 0.999 | (4.144, | (5.556, | 3.046 |
| | | | | | | 4.518, | 10.377, | |
| | | | | | | 7.19) | 35.345) | |
| G1167A | February 2021 | 117 | 7.265 | 43.59 | 0.977 | (4.518, | (10.377, | 2.747 |
| | | | | | | 7.19, | 35.345, | |
| | | | | | | 7.265) | 43.59) | |
| ΔR246 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔR246I | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| D253N | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| D253N | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔS247 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔS24 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔΥ248 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔΥ248 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔG252 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔG252 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔP251 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔP251 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔL249 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔL249 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔT250 | March 2021 | 302 | 8.632 | 30.795 | 0.99 | (7.19, | (0.862, | 1.442 |
| | | | | | | 7.265, | 6.838, | |
| | | | | | | 8.632) | 30.795) | |
| ΔT250 | April 2021 | 227 | 11.218 | 33.04 | 0.812 | (7.265, | (6.838, | 3.953 |
| | | | | | | 8.632, | 30.795, | |
| | | | | | | 11.218) | 33.04) | |
| ΔG72 | April 2021 | 227 | 11.218 | 5.286 | 0.948 | (7.265, | (0.855, | 3.953 |
| | | | | | | 8.632, | 3.642, | |
| | | | | | | 11.218) | 5.286) | |
| ΔI68 | April 2021 | 227 | 11.218 | 5.286 | 0.948 | (7.265, | (0.855, | 3.953 |
| | | | | | | 8.632, | 3.642, | |
| | | | | | | 11.218) | 5.286) | |
| ΔT73 | April 2021 | 227 | 11.218 | 5.286 | 0.948 | (7.265, | (0.855, | 3.953 |
| | | | | | | 8.632, | 3.642, | |
| | | | | | | 11.218) | 5.286) | |

### INCORPORATION BY REFERENCE

Each publication and patent mentioned herein is hereby incorporated by reference in its entirety. In case of conflict, the present specification, including any definitions herein, will control.

### EQUIVALENTS

While specific embodiments of the subject invention have been discussed, the above specification is illustrative and not restrictive. Many variations of the invention will become apparent to those skilled in the art upon review of the preceding description and the following claims. The full scope of the invention should be determined by reference to the claims, along with their full scope of equivalents, and by reference to the rest of the specification, along with such variations.

What is claimed is:

1. A composition for use as a vaccine against SARS-CoV-2 infection, comprising either one or more polypeptides that each comprise at least one surge-associated mutation in its amino acid sequence with respect to SEQ ID NO: 1 or one or more nucleic acids that encodes said one or more polypeptides.

2. The composition of claim 1, wherein said at least one surge-associated mutation is within the residue range 13-303 of SEQ ID NO: 1.

3. The composition of claim 1, wherein said at least one mutation is a deletion of any one or more residues selected from 14-16, 24, 67-74, 85-90, 138-146, 156-164, 167-174, 210-211, and 241-252.

4-5. (canceled)

6. The composition of claim 1, wherein said nucleic acid is a messenger ribonucleic acid (mRNA).

7. (canceled)

8. The composition of claim 6, wherein the mRNA comprises at least one non-canonical nucleobase.

9-11. (canceled)

12. The composition of claim 9, wherein the mutation comprises a contiguous stretch of residues.

13. The composition of claim 9, wherein the mutation comprises two separate contiguous stretches of residues.

14. The composition of claim 9, wherein the mutation comprises three or more separate contiguous stretches of residues.

15. The composition of claim 1, wherein said mutation is a deletion of one or more residues selected from those described in FIGS. 1 to 6 and Tables 1 to 5 and the Example.

16. The composition of claim 1, wherein said polypeptide also has K986P and V987P mutations.

17. The composition of claim 1, wherein said polypeptide has at least one additional mutation selected from E484K, N501Y, D614G, P681H, and P681R.

18. A composition comprising two or more of the polypeptides as defined in claim 1.

19. An antibody or an antigen-binding fragment thereof that binds to the polypeptide as defined in claim 1.

20. A formulation comprising at least one polypeptide as defined in claim 1 and at least one excipient.

21. (canceled)

22. The formulation of claim 20, further comprising a delivery system selected from protamine, protamine liposome, polysaccharide particles, cationic nanoemulsion, cationic polymer, cationic polymer liposome, cationic lipid nanoparticle, cationic lipid/cholesterol nanoparticles, cationic lipid/cholesterol/PEG nanoparticles, and dendrimer nanoparticles.

23-24. (canceled)

25. A method of vaccinating a subject against SARS-CoV-2 infection, comprising administering to the subject a composition of claim 1.

26. The method of claim 25, wherein said administering is via intramuscular injection or intradermal injection.

27. A method of selecting an antibody for treating a SARS-CoV-2 infection in a subject, comprising determining the presence of one or more mutations at a residue selected from 14-16, 24, 67-74, 85-90, 138-146, 156-164, 167-174, 210-211, and 241-252 in a SARS-CoV-2 spike protein from the subject; and selecting an antibody that does not bind to the N-terminal domain antigenic supersite of the SARS-CoV-2 spike protein.

28. A method of making an antibody, comprising using a polypeptide as defined in claim 1 as the target antigen.

29. A method of selecting a convalescent plasma against SARS-CoV-2 infection in a subject, comprising determining the presence of one or more mutations at a residue selected from 14-16, 24, 67-74, 85-90, 138-146, 156-164, 167-174, 210-211, and 241-252 in a SARS-CoV-2 spike protein from the subject; and selecting a convalescent plasma having antibodies that do not bind to the N-terminal domain antigenic supersite of the SARS-CoV-2 spike protein.

30. A method of selecting a vaccine against SARS-CoV-2 infection in a subject, comprising determining the presence of one or more mutations at a residue selected from 14-16, 24, 67-74, 85-90, 138-146, 156-164, 167-174, 210-211, and 241-252 in a spike protein from an emerging variant of SARS-CoV-2; and selecting a vaccine having a polypeptide of claim 1.
